(19) United States
(12) Patent Application Publication
(10) Pub. No.: US 2026/0201270 A1
(43) Pub. Date: Jul. 16, 2026

(54) ESTERS AND ETHERS OF 5-ISOPROPYL-2-METHYLCYCLOHEXAN-1-OL AS FRAGRANCE INGREDIENTS

(71) Applicant: BASF SE, Ludwigshafen am Rhein (DE)

(72) Inventors: Fabian HUNDEMER, Ludwigshafen am Rhein (DE); Christoph STOCK, Ludwigshafen am Rhein (DE); Ralf PELZER, Ludwigshafen am Rhein (DE); Florian GARLICHS, Ludwigshafen am Rhein (DE); Miriam BRU ROIG, Ludwigshafen am Rhein (DE)

(21) Appl. No.: 19/134,431

(22) PCT Filed: Dec. 1, 2023
(86) PCT No.: PCT/EP2023/083978

§ 371 Date: May 30, 2025

(30) Foreign Application Priority Data
Dec. 2, 2022 (EP) 22211086.8

(51) Int. Cl. C11B 9/00; A61K 8/33; A61K 8/37; A61K 8/49; A61Q 13/00; C07C 43/188; C07C 69/013; C07D 493/08; C11D 3/20; C11D 3/50

(52) U.S. Cl. CPC C11B 9/0076; A61K 8/33; A61K 8/37; A61K 8/4973; A61Q 13/00; C07C 43/188; C07C 69/013; C07D 493/08; C11B 9/0034; C11D 3/2068; C11D 3/2096; C11D 3/50; C07C 2601/14; C07C 2601/16

(57) ABSTRACT

The present invention relates to the use of a compound of formula (1) or of its mixtures with a compound of formula (II)

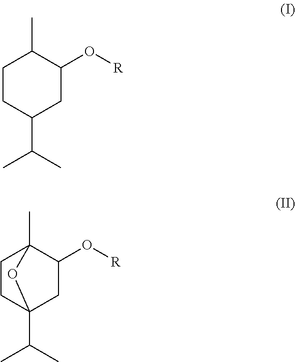

where R and the dotted line are as defined in the claims and the description, as aroma ingredient, to the use thereof to impart an aroma to a composition, and to a method of imparting an aroma to a composition. The present invention further relates to a composition comprising the compound of formula (I) or a mixture thereof with compound (II), to mixtures of the compound of formula (I) with the compound (II), and to specific compounds of formula (I).

### FIELD OF INVENTION

The present invention relates to the use of esters and ethers of 5-isopropyl-2-methylcyclohexan-1-ol of formula (I) as defined below and their mixtures with a compound of formula (II) as defined below as aroma ingredient, or for imparting an aroma to a composition, or for enhancing and/or modifying the aroma of a composition. The present invention is further directed to a composition comprising the compound of formula (1) or the mixture of compound of formula (I) and compound of formula (II), to mixtures of the compound of formula (I) with the compound (II), and to specific compounds of formula (I).

### BACKGROUND

Aroma chemicals, especially fragrances, are of great interest, especially in the field of cosmetics, cleaning and laundry compositions.

Despite a large number of already existing synthetic aroma chemicals, there is a constant need for new components in order to be able to satisfy the multitude of properties desired for extremely diverse areas of application. These include, firstly, the sensory properties, i.e. the compounds should have advantageous odiferous (olfactory) properties. Furthermore, aroma chemicals should also have additional positive secondary properties, such as e.g. an efficient preparation method, the possibility of providing better sensory profiles as a result of synergistic effects with other aroma chemicals, a higher stability under certain application conditions, and/or a higher substantivity.

Such properties are of special interest for compositions such as for example, care compositions, hygiene articles, cleaning compositions, textile detergent compositions and compositions for scent dispensers.

Of special interest are aroma chemicals which can impart one or more distinct sensory impressions to a composition, thereby contributing to a rich and interesting sensory profile, especially an olfactory profile of the composition. In this regard, aroma chemicals which can impart a fresh note, minty note, moldy note, carrot like note, natural note, rooty note, violet note, powdery note, floral note, woody note, warm note, ionone note, fruity note, camphoraceous note, citrus note, herbal note, ethereal note, gourmand note, anisic note, ambrinol like note, animalic note, civet note, soft note, incense note, musty note, fatty note, tobacco note, green note, spicy note, agrumic note, juniper note, pepper note, spearmint note, cedarwood note, dandelion note, earthy note, food-like note or any combination of two or more of these notes are of major interest. In addition, the substantivity as well as the tenacity are of special interest in order to obtain a long-lasting odiferous impression in the composition as well as to the surface which is treated with the composition.

However, since even small changes in chemical structure bring about massive changes in the sensory properties such as odor and/or flavor, the targeted search for substances with certain and distinct sensory properties such as a certain odor is extremely difficult. The search for new aroma chemicals is therefore in most cases difficult and laborious without knowing whether a substance with the desired odor and/or flavor will even actually be found.

It is an object of the present invention to provide substances which can be used as an aroma chemical either alone or as mixtures in compositions; in particular odor-intensive substances having a pleasant odor are sought. Furthermore, they should be combinable with other aroma chemicals, allowing the creation of novel advantageous sensory profiles and can be used in compositions.

It is an object of the present invention to provide a new aroma chemical or a mixture of aroma chemicals which has pleasant aroma, in particular a pleasant olfactory impression; preferably one or a combination of two or more of the impressions which are selected from the group of fresh note, minty note, moldy note, carrot like note, natural note, rooty note, violet note, powdery note, floral note, woody note, warm note, ionone note, fruity note, camphoraceous note, citrus note, herbal note, ethereal note, gourmand note, anisic note, ambrinol like note, animalic note, civet note, soft note, incense note, musty note fatty note, tobacco note, green note, spicy note, agrumic note, juniper note, pepper note, spearmint note, cedarwood note, dandelion note, earthy note, food-like note.

A further object of the present invention is that the aroma chemicals should be obtainable from readily available starting materials, allowing their fast and economic manufacturing. This is of special interest while using the aroma chemicals in compositions such as care compositions, hygiene articles, cleaning compositions, textile detergent compositions and compositions for scent dispensers.

### SUMMARY

These objects were achieved by the provision of the compound of formula (I) and its mixtures with compounds (II). It was surprisingly found that the compound of formula (I) and its mixtures according to the present invention have pleasant olfactory impression; where specific compounds (I) or mixtures of specific compounds (I) with specific compounds (II) (as described below) show an impression, preferably a combination of two or more impressions, which is/are selected from a fresh note, minty note, moldy note, carrot like note, natural note, rooty note, violet note, powdery note, floral note, woody note, warm note, ionone note, fruity note, camphoraceous note, citrus note, herbal note, ethereal note, gourmand note, anisic note, ambrinol like note, animalic note, civet note, soft note, incense note, musty note, fatty note, tobacco note, green note, spicy note, agrumic note, juniper note, pepper note, spearmint note, cedarwood note, dandelion note, earthy note, food-like note.

Thus, the first aspect of the present invention relates to the use of at least one compound of formula (I)

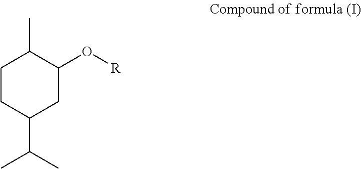

or of a stereoisomer thereof or of a mixture of different stereoisomers thereof,
where
---- (i.e. the interrupted bond/dotted line) represents a single bond or a double bond, and
R is linear or branched C₁-C₆-alkyl, formyl, or linear or branched C₁-C₄-alkylcarbonyl,
or of a mixture of the compound of formula (I) with a compound of formula (II) as defined bellow
as an aroma chemical.

Another aspect of the present invention relates to the use of the compound of formula (I) or of a mixture of the compound (I) with a compound (II) as defined below to impart an aroma impression to a composition.

Another aspect of the present invention relates to the use of a compound of formula (I) or of a of a mixture of the compound (I) and a compound (II) as defined below for intensifying and/or modifying the aroma character of a composition.

A further aspect of the present invention relates to a method of imparting an aroma impression to a composition comprising at least the step of adding the compound of formula (I) or the aforesaid mixture of (I) and (II) to a composition to be aromatized, or mixing at least one compound of formula (I) or the aforesaid mixture of (I) and (II) simultaneously or consecutively with the other components of said composition, or mixing at least one compound of formula (I) or the aforesaid mixture of (I) and (II) with a pre-formed mixture of a part of the other components of said composition.

A further aspect of the present invention relates to a method of boosting the aroma of a composition. Said method comprises the step of adding at least one compound of formula (I) or the aforesaid mixture of (I) and (II) to a composition the aroma of which is to be boosted, or mixing the at least one compound of formula (I) as defined above or the aforesaid mixture of (I) and (II) simultaneously or consecutively with the other ingredients of the composition, such as, e.g., at least one other aroma chemical different from the compound of formula (I) and (II) and/or at least one non-aroma chemical carrier, or mixing at least one compound of formula (I) as defined above or the aforesaid mixture of (I) and (II) with a pre-formed mixture of a part of the other components of said composition, so as to obtain the aroma composition.

Yet another aspect of the present invention relates to a method of intensifying and/or modifying the aroma of a composition. Said method comprises the step of incorporating at least one compound of formula (I) or the aforesaid mixture into an aroma composition so as to obtain an aroma-modified aroma composition, e.g. by taking the steps mentioned above for the other methods of the invention.

A further aspect of the present invention relates to a mixture comprising at least one compound of formula (I)

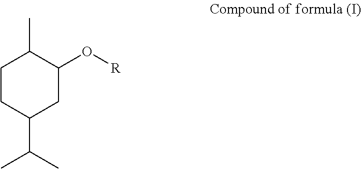

or a stereoisomer thereof or a mixture of different stereoisomers thereof,
where
---- represents a single bond or a double bond, and
R is linear or branched C₁-C₆-alkyl, formyl, or linear or branched C₁-C₄-alkylcarbonyl; and
at least one compound of formula (II)

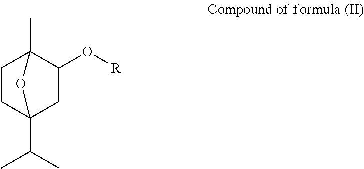

or a stereoisomer thereof or a mixture of different stereoisomers thereof, where
R is linear or branched C₁-C₆-alkyl, formyl, or linear or branched C₁-C₄-alkylcarbonyl.

A further aspect relates to a composition comprising at least one compound of formula (I) as defined above or a mixture of at least one compound of formula (I) and at least one compound of formula (II) as defined above, and
i. at least one aroma chemical (X) different from the compounds of formula (I) (and (II), or
ii at least one non-aroma chemical carrier, or
iii both of (i) and (ii).

Another aspect of the present invention relates to a compound (Ia)

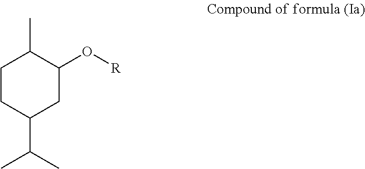

or a stereoisomer thereof or a mixture of different stereoisomers thereof, wherein R is isopropylcarbonyl.

Another aspect of the present invention relates to a compound of formula (Ib)

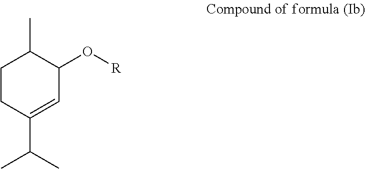

or a stereoisomer thereof or a mixture of different stereoisomers thereof,
where
R is linear or branched C₁-C₆-alkyl; where R is preferably selected from methyl, ethyl, propyl, isopropyl, n-butyl, isobutyl, tert-butyl, pentyl or hexyl, where more preferably R is methyl or ethyl, and is specifically ethyl.

Furthermore, compound of formula (I), compound of formula (II), or mixtures thereof, can be produced in good yields and purities by a simple synthesis starting from readily available starting materials and can be produced in large scales and in a simple and cost-efficient manner.

### DETAILED DESCRIPTION

Reference throughout this specification to “one embodiment” or “an embodiment” or “preferred embodiment” means that a particular feature, structure or characteristic described in connection with the embodiment is included in at least one embodiment of the present invention. Thus, appearances of the phrases “in one embodiment” or “in a preferred embodiment” in various places throughout this specification are not necessarily all referring to the same embodiment but may refer. Furthermore, the features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the subject matter, and form different embodiments, as would be understood by those in the art. For example, in the appended claims, any of the claimed embodiments are used in any combination.

Furthermore, the ranges defined throughout the specification include the end values as well, i.e. a range of 1 to 10 implies that both 1 and 10 are included in the range. For the avoidance of doubt, the applicant shall be entitled to any equivalents according to applicable law.

In the context of the present invention, the term “aroma” refers to a sensory property and comprises an odor and/or a flavor. Sensory property is related to the sensory perception of humans.

The term “aroma chemical” denotes a substance which is used to obtain a sensory or organoleptic (used interchangeably herein) impression and comprises its use to obtain an olfactory and/or a flavor impression. The term “olfactory impression” or “olfactory note” (used interchangeably here) denotes an odor impression without any positive or negative judgement, while the term “scent impression” or “fragrance impression” (used interchangeably herein) as used herein is connected to an odor impression which is generally felt as pleasant. Thus a “fragrance” or “scent” denotes an aroma chemical which predominately induces a pleasant odor impression. A flavor denotes an aroma chemical which induces a taste impression.

The term “aroma composition”, as used herein, refers to a composition which induces or has an aroma. The term aroma composition comprises “odor composition” and/or “flavor composition”. An odor composition is a composition which predominately induces an odor impression, and a flavor composition is a composition which predominantly induces a taste impression.

The term “aroma profile” denotes the overall aroma impression of an aroma chemical and is composed of the individual aroma impressions of an aroma chemical.

The term odor composition comprises “fragrance composition” or “scent composition” (used interchangeably herein), where “fragrance composition” or “scent composition” predominately induce an odor impression which is generally felt as pleasant.

The general expressions “advantageous sensory properties” or “advantageous organoleptic properties” describe the niceness and conciseness of an organoleptic impression conveyed by an aroma chemical. “Niceness” and “conciseness” are terms which are familiar to the person skilled in the art, such as a perfumer. Niceness generally refers to a spontaneously brought about, positively perceived, pleasant sensory impression. However, “nice” does not have to be synonymous with “sweet”. “Nice” can also be the odor of musk or sandalwood. “Conciseness” generally refers to a spontaneously brought about sensory impression which—for the same test panel-brings about a reproducibly identical reminder of something specific. For example, a substance can have an odor which is spontaneously reminiscent of that of an “apple”: the odor would then be concisely of “apples”. If this apple odor were very pleasant because the odor is reminiscent, for example, of a sweet, fully ripe apple, the odor would be termed “nice”.

However, the odor of a typically tart apple can also be concise. If both reactions arise upon smelling the substance, in the example thus a nice and concise apple odor, then this substance has particularly advantageous sensory properties.

The expressions “combination of”, “in combination with” or “combined with” when used herein referring to the compositions, methods or the use of two compounds, take account of the fact that the two compounds do not need to be used in the form of a physical mixture of said compounds but can be used (e.g., added) separately. Where the compounds are used separately, they can be used (e.g. added) sequentially (i.e. one after the other) in any order, or concurrently (i.e. basically at the same time).

The term “boosting”, or “boost” is used herein to describe the effect of enhancing and/or modifying the aroma of an aroma chemical or of a composition, respectively of an aroma composition. The term “enhancing” comprises an improvement of the niceness and/or conciseness of an aroma and/or an improvement of the intensity. The term “modifying” comprises the change of an aroma profile. The terms “niceness” and “conciseness” are familiar to the person skilled in the art, such as a perfumer and have the respective meaning.

The intensity can be determined via a threshold value determination. A threshold value of an odor is the concentration of a substance in the relevant gas space at which an odor impression can just still be perceived by a representative test panel, although it no longer has to be defined.

Booster effects are particularly desired in fragrance composition when top-note-characterized applications are required, in which the odor is to be conveyed particularly quickly and intensively, for example in deodorants, air fresheners or in the taste sector in chewing gums.

The terms “the invention relates to” and “the invention is directed to” are used synonymously throughout the invention.

The term “tenacity” describes the evaporation behavior over time of an aroma chemical. The tenacity can for example be determined by applying the aroma chemical to a test strip, and by subsequent olfactory evaluation of the odor impression of the test strip. For aroma chemicals with high tenacity the time span after which the panel can still identify an aroma impression is long.

The term “substantivity” describes the interaction of an aroma chemical with a surface, such as for example the skin or a textile, especially after subsequent treatment of the surface, such as for example washing. The substantivity can for example be determined by washing a textile with a textile detergent composition comprising the aroma chemical and subsequent olfactory evaluation of the textile directly after washing (wet textile) as well as evaluation of the dry textile after prolonged storage.

The term “stability” describes the behavior of an aroma chemical upon contact with oxygen, light and/or other substances. An aroma chemical with high stability maintains its aroma profile over a long period in time, preferably in a large variety of compositions and under various storage conditions.

In order to impart a long-lasting aroma impression to a composition or to a surface treated with a composition, the tenacity, the substantivity as well as the stability of the aroma chemical in the compositions should preferably be high.

As used herein the terms “compound (I)” or “compounds (I)” refer to the compound(s) of formula (I) including all the stereoisomeric forms (stereoisomers) thereof in all ratios and the salts thereof. The term “compound (I)” or “compound of formula (I)” or just “(I)” is used interchangeably. This applies to compound of formula (II) also.

The term “stereoisomer” is a general term used for all isomers of individual compounds that differ only in the orientation of their atoms in space, not in the connectivity of the atoms Thus, the term stereoisomer includes mirror image isomers (enantiomers), geometric (cis/trans or E/Z) isomers, and diastereoisomers. For precise definitions of the terms, see G. Helmchen: “Vocabulary and Nomenclature of Organic Stereochemistry”, in Houben-Weyl E21a, Stereoselective Synthesis G. Helmchen, R.W. Hoffmann, J. Mulzer, E. Schaumann (Editors), 1995, 1-74. The possible isomers can be present as mixtures (i.e. racemates, cis/trans-mixtures or mixtures of diasteroisomers).

In the present case, compounds (I), wherein the dotted line (----) is a single bond, have at least three stereogenic centers, namely the carbon ring atoms carrying the methyl, the isopropyl and the —OR group. Compounds (I), wherein the dotted line is a double bond, have at least two stereogenic centers, namely the carbon ring atoms carrying the methyl and the —OR group. Moreover, if R is or contains a branched C₄-C₆-alkyl, R can also contain one or more stereogenic centers (as, for example, in sec-butyl).

In terms of the present invention, the term “pure enantiomer” is understood as a non-racemic mixture of a specific compound, where the desired enantiomer is present in an enantiomeric excess of >90% ee.

In terms of the present invention, the term “pure diastereomer” is understood as a mixture of the diastereomers of a specific compound, where the desired diastereomer is present in an amount of >90%, based on the total amount of diastereomers of said compound.

In the present context, the term “(I)”, “compound (I)” or “compound of formula (I)”, when not defined as a specific stereoisomer or a specific mixture of stereoisomers, refers to the form of the compound as it is obtained in a non-stereoselective method used for its production. The term is however also used if it is not necessary or not possible to specify in more detail the stereochemistry of the compound (I). The same applies to compound (II).

The term “alkyl” denotes in each case a straight-chain (=linear) or branched alkyl group having the indicated number of carbon atoms. Examples of C₁-C₂-alkyl are methyl and ethyl. Examples of C₁-C₃-alkyl are methyl, ethyl, n-propyl and isopropyl. Examples of C₁-C₄-alkyl are methyl, ethyl, n-propyl, isopropyl, n-butyl, 2-butyl (=sec-butyl), isobutyl and tert-butyl. Examples of C₁-C₆-alkyl are methyl, ethyl, n-propyl, isopropyl, n-butyl, 2-butyl (=sec-butyl), isobutyl, tert-butyl, n-pentyl, 1-methylbutyl, 2-methylbutyl, 3-methylbutyl, 1,1-dimethylpropyl, 2,2-dimethylpropyl, 1,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, and (other) structural isomers thereof.

Formyl is a group —CH(═O).

Linear or branched C₁-C₄-alkylcarbonyl is a group —C(═O)—R′, where R′ is a linear or branched C₁-C₄-alkyl. Examples are methylcarbonyl (acetyl), ethylcarbonyl (propionyl), n-propylcarbonyl, isopropylcarbonyl, n-butylcarbonyl, 2-butylcarbonyl (=sec-butylcarbonyl), isobutylcarbonyl and tert-butylcarbonyl.

### Use:

One embodiment the present invention relates to the use of at least one compound of formula (I)

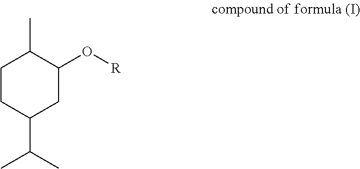

or of a stereoisomer thereof or of a mixture of different stereoisomers thereof,
where
---- represents a single bond or a double bond, and
R is linear or branched C₁-C₆-alkyl, formyl, or linear or branched C₁-C₄-alkylcarbonyl, as an aroma chemical, or to impart an aroma impression to a composition.

In a preferred embodiment, the present invention relates to the use of at least one compound of formula (I), wherein compound of formula (I) is compound of formula (Ia) (which is a compound (I) wherein the dotted line (----) represents a single bond)

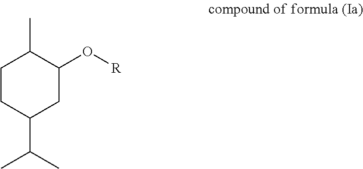

or of a stereoisomer thereof or of a mixture of different stereoisomers thereof,
where
R is linear or branched C₁-C₆-alkyl, formyl, or linear or branched C₁-C₄-alkylcarbonyl, as an aroma chemical, or to impart an aroma impression to a composition.

In another embodiment, the present invention relates to the use of at least one compound of formula (I), wherein compound of formula (I) is compound of formula (Ib) (which is a compound (I) wherein the dotted line (----) represents a double bond),

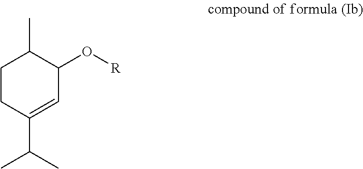

or stereoisomer thereof or a mixture of different stereoisomers thereof,
where
R is linear or branched C₁-C₆-alkyl, formyl, or linear or branched C₁-C₄-alkylcarbonyl, as an aroma chemical, or to impart an aroma impression to a composition.

Among compounds (Ia) and (Ib), preference is given to compounds (Ia).

In a preferred embodiment, R is linear or branched C₁-C₆-alkyl, where C₁-C₆-alkyl is preferably selected from methyl, ethyl, propyl, isopropyl, n-butyl, isobutyl, tert-butyl, pentyl or hexyl.

More preferably, R is methyl or ethyl.

In another preferred embodiment, R is formyl, or linear or branched C₁-C₄-alkylcarbonyl.

In a more preferred embodiment, R is linear or branched C₁-C₄-alkylcarbonyl, where C₁-C₄-alkylcarbonyl is preferably selected from methylcarbonyl, ethylcarbonyl, n-propylcarbonyl, isopropylcarbonyl, n-butylcarbonyl, isobutylcarbonyl and tert-butylcarbonyl.

More preferably, R is C₁-C₃-alkylcarbonyl. Even more preferably, R is methylcarbonyl, ethylcarbonyl or isopropylcarbonyl. Particularly preferably, R is methylcarbonyl or ethylcarbonyl.

In a particularly preferred embodiment, the compound of formula (I) is a compound of formula (Ia), wherein R is linear or branched C₁-C₃-alkylcarbonyl, in particular methylcarbonyl, ethylcarbonyl or isopropylcarbonyl and is more particularly methylcarbonyl or ethylcarbonyl.

A preferred embodiment of the present invention is directed to the use of at least one compound of formula (I) as aroma chemical to impart a fresh note, minty note, moldy note, carrot like note, natural note, rooty note, violet note, powdery note, floral note, woody note, warm note, lonone note, fruity note, camphoraceous note, citrus note, herbal note, ethereal note, gourmand note, anisic note, ambrinol like note, animalic note, civet note, soft note, incense note, musty note, fatty note, tobacco note, green note, agrumic note, or a combination of two or more of these notes.

In an embodiment, the compound of formula (I) includes the compound of formula (Ia) and compound of formula (Ib).

In a further embodiment, the present invention is directed to the use of at least one compound of formula (I) to boost the aroma of a composition.

In a preferred embodiment of the present invention, the compound of formula (I) is used as a fragrance or as constituent of a fragrance composition or to impart a fragrance to a composition.

In particular, the at least one compound of formula (I) is used to impart a note that is selected from a fresh note, minty note, moldy note, carrot like note, natural note, rooty note, violet note, powdery note, floral note, woody note, warm note, ionone note, fruity note, camphoraceous note, citrus note, herbal note, ethereal note, gourmand note, anisic note, ambrinol like note, animalic note, civet note, soft note, incense note, musty note, fatty note, tobacco note, green note, agrumic note or a combination of two or more of these notes to a composition.

More particularly, the at least one compound of formula (I) is used to impart a combination of two or more of the impression which are selected from fresh note, minty note, moldy note, carrot like note, natural note, rooty note, violet note, powdery note, floral note, woody note, warm note, ionone note, fruity note, camphoraceous note, citrus note, herbal note, ethereal note, gourmand note, anisic note, ambrinol like note, animalic note, civet note, soft note, incense note, musty note, fatty note, tobacco note, green note, agrumic note or a combination of two or more of these notes to a composition.

Suitable compositions are for example compositions used in personal care, in home care, in industrial applications as well as compositions used in other applications, such as pharmaceutical compositions or crop protection compositions.

Preferably, compounds of formula (I) according to the present invention are used in a composition selected from perfume compositions, body care compositions (including cosmetic compositions and products for oral and dental hygiene), hygiene articles, cleaning compositions (including dishwashing compositions), textile detergent compositions, compositions for scent dispensers, foods, food supplements, pharmaceutical compositions, or crop protection compositions. Further details are given below in context with the composition of the invention.

Compounds (I) are obtainable by standard methods of organic chemistry, generally by the etherification (alkylation) or esterification (acylation) of the respective alcohol 5-isopropyl-2-methylcyclohexan-1-ol (for preparing compounds (Ia)) or 3-isopropyl-6-methylcyclohex-2-en-1-ol (for preparing compounds (Ib)).

Suitable etherification and esterification conditions are known in the art.

The etherification can be carried out by reacting said alcohol with an alkylation agent R-X, where R is C₁-C₆-alkyl and X represents a leaving group, such as a halogen atom, e.g. C₁, Br or I, or a sulfonate group, e.g. tosylate, mesylate, triflate or nonaflate, typically in the presence of a base. Suitable bases can be inorganic bases or organic bases. Suitable inorganic bases are for example alkali metal carbonates, e.g. Li₂CO₃, Na₂CO₃, K₂CO₃ or Cs₂CO₃, alkali metal hydroxides, e.g. LiOH, NaOH or KOH, and hydride donors, e.g. NaH, LiAIH₄ or NaBH₄. Examples for suitable organic bases are tertiary amines, e.g. trimethylamine, triethylamine, tripropylamine, ethyldiisopropylamine and the like, or basic N-heterocycles, such as morpholine, pyridine, lutidine, DMAP, DABCO, DBU or DBN. Particularly expedient is NaH, since hydrogen formed in the reaction thereof with the alcohol drives the reaction even stronger.

The alkylation reaction is performed under conventional alkylation reaction conditions that are well known to the skilled person.

The reaction can be carried out in a solvent or neat. Examples for suitable solvents are listed below in context with the esterification reaction.

After completion of the reaction, the etherification product can be isolated by usual means, if necessary after neutralization (especially if X is a halogen atom or if acidic conditions were applied), for example by distillative, extractive or chromatographic methods. If desired, the isolated product can subsequently be further purified.

The esterification can be carried out by reacting said alcohol (i.e. 5-isopropyl-2-methylcyclohexan-1-ol for preparing compounds (Ia) or 3-isopropyl-6-methylcyclohex-2-en-1-ol for preparing compounds (Ib)) with the desired acid R—C(O)OH, where R is H or C₁-C₄-alkyl, i.e. with formic acid, acetic acid, propionic acid, butyric acid, isobutyric acid or one of the pentanoic acids, or with a more reactive derivative thereof, e.g. a halide thereof (especially the chloride) or an anhydride thereof, under typical esterification conditions; if desired under removal of the reaction water formed (if the acid as such is used) or of the acid formed (if an anhydride is used) to enhance the reaction rate, or under neutralization (if an acid halide is used). The reaction is generally carried out in the presence of an esterification catalyst. Suitable esterification catalysts that can be applied in this reaction are well known to the skilled person. Suitable esterification catalysts are for example metal based catalysts, e.g. iron, cadmium, cobalt, lead, zinc, antimony, magnesium, titanium and tin catalysts in the form of metals, metal oxides or metal salts, such as metal alkoxylates; or acids, such as mineral acids, e.g. sulfuric acid, hydrochloric acid or phosphoric acid; organic sulfonic acids, e.g. methane sulfonic acid or para-toluene sulfonic acid, or acidic ion exchangers.

The reaction of the acid halide R—C(O)—X (where X is a halogen atom) with the alcohol is generally carried out in the presence of a base. Suitable bases are preferably selected from organic bases. Suitable organic bases that can be used are for example tertiary amines, e.g. trimethylamine, triethylamine, tripropylamine, ethyldiisopropyl-amine and the like, or basic N-heterocycles, such as morpholine, pyridine, lutidine, DMAP, DABCO, DBU or DBN.

The reaction of the acid anhydride is also generally carried out in the presence of a base. Suitable bases are listed above. Specific preference is here given to DMAP.

Alternatively, esterification can be carried out as a Steglich esterification using a coupling reagent, such as N,N′-di(propan-2-yl) methanediimine (DIC) or dicyclohexylcarbodiimide (DCC), and 4-dimethylaminopyridine (DMAP) as a catalyst.

The esterification reaction can be carried out in a solvent or neat. Suitable solvents are e.g. alkanes, such as hexane or heptane, halogenated C₁-C₄-alkanes, such as dichloromethane, chloroform or dichloroethane, cycloalkanes, such as cyclohexane, aromatic hydrocarbons, such as toluene, the xylenes, chlorobenzene or dichlorobenzene, aliphatic ethers, such as diisopropylether or methyl-tert-butyl ether, cyclic ethers, such as tetrahydrofuran or the dioxanes, and the like. The specified solvents can be used on their own or in the form of mixtures with one another.

After completion of the reaction, the esterification product can be isolated by usual means, if necessary after neutralization (especially if an acid halide or anhydride was used as starting material or if the acid was used in excess) and/or removal of the catalyst (especially if this is solid, e.g. one of the above-mentioned metal based catalysts or ionic exchange resins), for example by distillative, extractive or chromatographic methods. If desired, the isolated product can subsequently be further purified.

The alcohols are in turn obtainable from 3-isopropyl-6-methylcyclohex-2-en-1-one (carvenone), which is commercially available. The keto group thereof can be reduced selectively to afford 3-isopropyl-6-methylcyclohex-2-en-1-ol; or 3-isopropyl-6-methylcyclohex-2-en-1-one can be subjected to a hydrogenation reaction where both the C—C and the C—O double bond are hydrogenated to afford 5-isopropyl-2-methylcyclohexan-1-ol. The selective reduction of the keto group (where the C═C double bond is not hydrogenated) can be carried out with suitable reduction agents, such as complex hydrides, e.g. LiAlH₄ (Iithium aluminum hydride) or NaBH₄. The hydrogenation reaction where both the C—C and the C—O double bond are reduced can be carried out under usual hydrogenation conditions, such as hydrogenation under elevated pressure, generally in the presence of a hydrogenation catalyst, such as group VIII metals, e.g. Pt, Pd or Ni. For preparing 5-isopropyl-2-methylcyclohexan-1-ol, it is alternatively possible to hydrogenate carvone (1-methyl-4-isopropenyl-6-cyclohex-1-en-2-one), which is also commercially available. Suitable hydrogenation conditions correspond to those for the unselective hydrogenation of carvenone.

### Compound:

One embodiment of the present invention relates to a compound of formula (Ia)

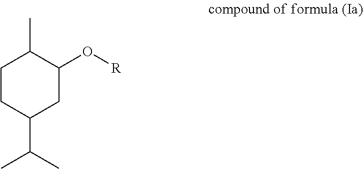

or a stereoisomer thereof or a mixture of different stereoisomers thereof,
wherein R is isopropylcarbonyl.

Another embodiment of the present invention relates to a compound of formula (Ib)

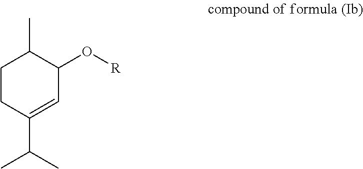

or stereoisomer thereof or a mixture of different stereoisomers thereof,
wherein
R is linear or branched C₁-C₆ alkyl.

Preferably, R is selected from methyl, ethyl, propyl, isopropyl, n-butyl, isobutyl, tert-butyl, pentyl or hexyl. More preferably, R is methyl or ethyl. Specifically, R is ethyl.

These compounds are obtainable with the method described below in context with the mixtures of the invention

### Mixture:

One embodiment of the present invention relates to a mixture comprising at least one compound of formula (I)

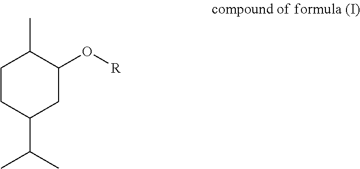

or a stereoisomer thereof or a mixture of different stereoisomers thereof,
where
---- represents a single bond or a double bond, and
R is linear or branched C₁-C₆-alkyl, formyl, or linear or branched C₁-C₄-alkylcarbonyl;
and
at least one compound of formula (II)

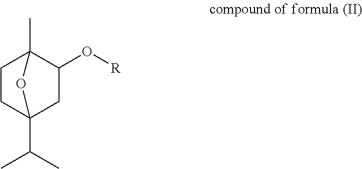

or a stereoisomer thereof or a mixture of different stereoisomers thereof,
where
R is linear or branched C₁-C₆-alkyl, formyl, or linear or branched C₁-C₄-alkylcarbonyl.

Preferably, in compounds (I) and (II) R has the same meaning.

The above remarks to preferred embodiments of compounds (I) apply also in the case of mixtures with compounds (II).

Thus, among compounds (Ia) and (Ib), preference is given to compounds (Ia).

In a preferred embodiment, R is linear or branched C₁-C₆-alkyl, where C₁-C₆-alkyl is preferably selected from methyl, ethyl, propyl, isopropyl, n-butyl, isobutyl, tert-butyl, pentyl or hexyl. More preferably, R is methyl or ethyl.

In another preferred embodiment, R is formyl, or linear or branched C₁-C₄-alkylcarbonyl. More preferably, R is linear or branched C₁-C₄-alkylcarbonyl, where C₁-C₄-alkylcarbonyl is preferably selected from methylcarbonyl, ethylcarbonyl, n-propylcarbonyl, isopropylcarbonyl, n-butylcarbonyl, isobutylcarbonyl and tert-butylcarbonyl. Even more preferably, R is C₁-C₃-alkylcarbonyl, where C₁-C₃-alkylcarbonyl is preferably methylcarbonyl, ethylcarbonyl or isopropylcarbonyl. Particularly preferably, R is methylcarbonyl or ethylcarbonyl.

In a particularly preferred embodiment, the compound of formula (I) is a compound of formula (Ia), wherein R is linear or branched C₁-C₃-alkylcarbonyl, in particular methylcarbonyl, ethylcarbonyl or isopropyl carbonyl, and is more particularly methylcarbonyl or ethylcarbonyl.

In a preferred embodiment, the mixture comprises compounds of formula (I) and compounds of formula (II) in a weight ratio in the range 0.01:99.99 to 99.99:0.01.

In a more preferred embodiment, the mixture comprises compounds of formula (I) and compounds of formula (II) in a weight ratio in the range of 100:1 to 1:100, e.g. the range of 97:1 to 3:97 or 94:5 to 5:94 or 10:90 to 90:10, or 11:89 to 89:11 or 12:88 to 88:12, or 13: 87 to 87:13, or 14:86 to 86:14, or 15:85 to 85:15, or 16:84 to 84:16, or 17:83 to 83:17, or 18:82 to 82:18, or 19:81 to 81:19, or 20:80 to 80:20.

Preferably, the amount of compound (I) exceeds that of compound (II). Thus, the weight ratio of the total amount of compounds of formula (I) to the total amount of compounds of formula (II) is particularly preferably in the range from 100:1 to 1:1, more particularly from 50:1 to 1:1, even more particularly from 35:1 to 2:1, and specifically from 25:1 to 3:1, e.g. 20:1 to 4:1.

Compounds (II) are available from the etherification or esterification of 2-hydroxy-1,4-cineole (a compound (II) wherein R is hydrogen, so to say).

2-Hydroxy-1,4-cineole is in turn obtainable from terpinen-4-ol, which is epoxidised at the double bond. Acid-catalyzed rearrangement of the epoxide formed in this reaction yields 2-hydroxy-1,4-cineole. This synthetic path is described in WO2023/144219, WO 2018/210662, EP 0081892 or EP 0081893 and the literature cited therein. In the acid-catalyzed rearrangement of the epoxide, 5-hydroxy-5-isopropyl-2-methylcyclohexan-1-one is formed as by-product, which can be dehydrated to yield carvenone (3-isopropyl-6-methylcyclohex-2-en-1-one; as described in context with the methods for obtaining compounds (I), this is the compound which can be reduced to the alcohol from which compounds (I) are prepared). Carvenone may contain residual amounts of 2-hydroxy-1,4-cineole. If 2-hydroxy-1,4-cineole is not separated from carvenone nor from the alcohol obtained by the above-described reduction thereof (either selectively at the carbonyl group or both at the carbonyl group and the C═C double bond), it is still present in the esterification or etherification reaction of the alcohol obtained in the carvenone reduction, where its OH group is acylated or alkylated alongside the OH group of said alcohol. This results in mixtures of compounds (I) and (II); R being the same in these compounds.

If 2-hydroxy-1,4-cineole is provided in individual form (and not as by-product in the above-described reaction), compounds (II) are obtainable by the etherification or esterification of 2-hydroxy-1,4-cineole. Suitable etherification or esterification conditions correspond to those described above for the synthesis of compounds (I). Compounds (II) can then be mixed with compounds (1) in any desired ratio; also R may vary in the compounds (I) and (II).

The mixture of at least one compound of formula I and at least one compound of formula II can thus be obtained directly from the reaction mixture or can be physically mixed in any desired ratio depending on the end use of the combination in any suitable composition.

Out of economic reasons, it is however advantageous to provide the mixture of compounds (I) and (II) from said reaction mixture.

A preferred embodiment of the present invention is directed to the use of mixture to impart a camphoraceous note, citrus note, floral note, herbal note, ethereal note, fresh note, ionone note, spicy note, woody note, carrot like note, juniper note, pepper note, warm note, spearmint note, cedarwood note, minty note, dandelion note, earthy note, food-like note or any combination of two or more of these notes to a composition.

A further embodiment is directed to a method of imparting an aroma impression to a composition comprising at least the step of adding the aforesaid mixture to a composition.

Another embodiment of the present invention relates to the mixture comprising at least one compound of formula (I), at least one compound of formula (II) to impart an aroma impression to a composition.

In a further embodiment the present invention is directed to the use of the aforesaid mixture to boost the aroma of a composition.

In an embodiment of the present invention, the mixture is used as a fragrance or as constituent of a fragrance composition.

Suitable compositions are for example compositions used in personal care, in home care, in industrial applications as well as compositions used in other applications, such as pharmaceutical compositions or crop protection compositions.

Preferably, the mixtures according to the present invention are used in a composition selected from perfume compositions, body care compositions (including cosmetic compositions and products for oral and dental hygiene), hygiene articles, cleaning compositions (including dishwashing compositions), textile detergent compositions, compositions for scent dispensers, foods, food supplements, pharmaceutical compositions, or crop protection compositions.

The compositions are described in more detail in the below paragraphs.

### Compound (II):

Some compounds (II) are novel and are therefore also part of the present invention. Thus, the invention relates to a compound of formula (II)

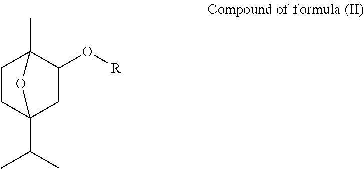

or a stereoisomer thereof or a mixture of different stereoisomers thereof,
wherein R is methyl, ethyl, ethylcarbonyl or isopropylcarbonyl.

### Composition:

In an embodiment, the present invention relates to a composition comprising compound of formula (I) or mixture comprising compound of formula (I) and compound of formula (II), and
(i) at least one aroma chemical (X) other than compound of formula I, compound of formula II, or stereoisomers thereof, or
(ii) at least one non-aroma chemical carrier, or
(iii) both of (i) and (ii).

Preferably, in this embodiment, the composition comprises the compound of formula (I) (if compound (II) is not present) or the mixture comprising the compound of formula (I) and the compound of formula (II) in a total amount in the range of ≥0.01 wt. % to ≤70.0 wt. %, based on the total weight of the composition.

Preferably, the composition is an aroma composition, more preferable a fragrance composition.

Aroma chemical different from compound of formula (I) and compound of formula (II):

In one embodiment, the composition comprises at least one aroma chemical which is different from the compound of formula (I) and from the compound of formula (II).

Aroma chemicals which are different from the compound of formula (I) and (II) are also referred to as aroma chemical (X).

By virtue of their physical properties, the compound of formula (I) or the mixture comprising the compound of formula (I) and the compound of formula (II) is well combinable with aroma chemicals which are different from the compound of formula (I) and from the compound of formula (II) and other customary ingredients in aroma compositions, in particular fragrance compositions. This allows, e.g., the creation of aroma compositions (preferably fragrance compositions) which have novel advantageous sensory profiles. Especially, as already explained above, the compounds can provide a booster effect for other aroma chemicals (such as fragrances).

The aroma chemical (X) is preferably selected from:

geranyl acetate, alpha-hexylcinnamaldehyde, 2 phenoxyethyl isobutyrate, dihydromyrcenol, methyl dihydrojasmonate, 4,6,6,7,8,8 hexamethyl-1,3,4,6,7,8-hexa-hydro-cyclopenta [g]benzopyran, tetrahydrolinalool, ethyllinalool, benzyl salicylate, 2 methyl-3-(4-tert-butylphenyl) propanal, cinnamyl alcohol, 4,7 methano-3a,4,5,6,7,7a-hexahydro-5 indenyl acetate and/or 4,7 methano-3a,4,5,6,7,7a-hexahydro-6-indenyl acetate, citronellol, citronellyl acetate, tetrahydrogeraniol, vanillin, linalyl acetate, styrolyl acetate, octahydro-2,3,8,8-tetramethyl-2-acetonaphthone and/or 2 acetyl-1,2,3,4,6,7,8-octahydro-2,3,8,8-tetramethylnaphthalene, hexyl salicylate, 4 tert-butylcyclohexyl acetate, 2-tert-butylcyclohexyl acetate, alpha-ionone, n alpha-methylionone, alpha-iso-methylionone, coumarin, terpinyl acetate, 2 phenylethyl alcohol, 4-(4-hydroxy-4-methylpentyl)-3-cyclohexene-carboxaldehyde, alpha-amylcinnamaldehyde, ethylene brassylate, (E) and/or (Z)-3-methylcyclopentadec-5 enone, 15-pentadec-11-enolide and/or 15-pentadec-12-enolide, 15-cyclopentadecanolide, 1-(5,6,7,8-tetrahydro-3,5,5,6,8,8-hexamethyl-2-naphthalenyl) ethanone, 2-isobutyl-4-methyltetrahydro-2H pyran-4-ol, 2-ethyl-4-(2,2,3-trimethyl-3-cyclopenten-1-yl)-2-buten-1-ol, cis-3-hexenyl acetate, trans-3-hexenyl acetate, trans-2/cis-6-nonadienol, 2,4-dimethyl-3-cyclohexenecarboxaldehyde, 2,4,4,7-tetramethyloct-6-en-3-one, 2,6-dimethyl-5-hepten-1-al, borneol, 3 (3 isopropylphenyl) butanal, 2-methyl-3-(3,4-methylenedioxyphenyl) propanal, 3-(4-ethylphenyl)-2,2-dimethylpropanal, 7-methyl-2H 1,5-benzodioxepin-3 (4H)-one, 3,3,5-trimethylcyclohexyl acetate, 2,5,5 trimethyl-1,2,3,4,4a,5,6,7-octahydronaphthalen-2-ol, 3-(4-tert-butylphenyl)-propanal, ethyl 2-methylpentanoate, ethoxymethoxycyclododecane, 2,4-dimethyl-4,4a,5,9b-tetrahydroindeno[1,2-d][1,3]dioxine, (2-tert-butylcyclohexyl)acetate, or 3-[5,5,6-trimethylbicyclo[2.2.1]hept-2-yl]cyclohexan-1-ol.

In a preferred embodiment, the at least one aroma chemical (X) is selected from methyl benzoate, benzyl acetate, geranyl acetate, 2-isobutyl-4-methyltetrahydro-2H-pyran-4-ol, linalool, 2-isobutyl-4-methyltetrahydro-2H-pyran-4-ol, or methyl benzoate.

In another preferred embodiment, the at least one aroma chemical (X) is selected from ethylvanillin, vanillin, 2,5-dimethyl-4-hydroxy-2H-furan-3-one (furaneol), or 3-hydroxy-2-methyl-4H-pyran-4-one (maltol).

Further aroma chemicals with which the compound of formula (I) can be combined to give a composition according to the present invention can be found, e.g., in S. Arctander, Perfume and Flavor Chemicals, Vol. I and II, Montclair, N. J., 1969, self-published or H. Surburg and J. Panten, Common Fragrance and Flavor Materials, 4th Ed., Wiley-VCH, Weinheim 2016. Specifically, mention may be made of:

extracts from natural raw materials such as essential oils, concretes, absolutes, resins, resinoids, balsams, tinctures such as e.g.

ambergris tincture; amyris oil; angelica seed oil; angelica root oil; aniseed oil; valerian oil; basil oil; tree moss absolute; bay oil; mugwort oil; benzoin resin; bergamot oil; beeswax absolute; birch tar oil; bitter almond oil; savory oil; buchu leaf oil; cabreuva oil; cade oil; calmus oil; camphor oil; cananga oil; cardamom oil; cascarilla oil; cassia oil; cassia absolute; castoreum absolute; cedar leaf oil; cedar wood oil; cistus oil; citronella oil; lemon oil; copaiba balsam; copaiba balsam oil; coriander oil; costus root oil; cumin oil; cypress oil; davana oil; dill weed oil; dill seed oil; Eau de brouts absolute; oak moss absolute; elemi oil; tarragon oil; eucalyptus citriodora oil; eucalyptus oil; fennel oil; pine needle oil; galbanum oil; galbanum resin; geranium oil; grapefruit oil; guaiacwood oil; gurjun balsam; gurjun balsam oil; helichrysum absolute; helichrysum oil; ginger oil; iris root absolute; iris root oil; jasmine absolute; calmus oil; camomile oil blue; roman camomile oil; carrot seed oil; cascarilla oil; pine needle oil; spearmint oil; caraway oil; labdanum oil; labdanum absolute; labdanum resin; lavandin absolute; lavandin oil; lavender absolute; lavender oil; lemongrass oil; lovage oil; lime oil distilled; lime oil pressed; linalool oil; litsea cubeba oil; laurel leaf oil; mace oil; marjoram oil; mandarin oil; massoia bark oil; mimosa absolute; musk seed oil; musk tincture; clary sage oil; nutmeg oil; myrrh absolute; myrrh oil; myrtle oil; clove leaf oil; clove flower oil; neroli oil; olibanum absolute; olibanum oil; opopanax oil; orange blossom absolute; orange oil; origanum oil; palmarosa oil; patchouli oil; perilla oil; peru balsam oil; parsley leaf oil; parsley seed oil; petitgrain oil; peppermint oil; pepper oil; pimento oil; pine oil; pennyroyal oil; rose absolute; rose wood oil; rose oil; rosemary oil; Dalmatian sage oil; Spanish sage oil; sandalwood oil; celery seed oil; spike-lavender oil; star anise oil; styrax oil; tagetes oil; fir needle oil; tea tree oil; turpentine oil; thyme oil; tolubalsam; tonka absolute; tuberose absolute; vanilla extract; violet leaf absolute; verbena oil; vetiver oil; juniper berry oil; wine lees oil; wormwood oil; winter green oil; hyssop oil; civet absolute; cinnamon leaf oil; cinnamon bark oil, and fractions thereof, or ingredients isolated therefrom;

individual fragrances from the group of hydrocarbons, such as e.g. 3 carene; alpha-pinene; beta-pinene; alpha-terpinene; gamma-terpinene; p-cymene; bisabolene; camphene; caryophyllene; cedrene; farnesene; limonene; longifolene; myrcene; ocimene; valencene; (E,Z)-1,3,5-undecatriene; styrene; diphenylmethane;

the aliphatic alcohols such as e.g. hexanol; octanol; 3-octanol; 2,6-dimethylheptanol; 2-methyl-2-heptanol; 2-methyl-2-octanol; (E)-2-hexenol; (E)-and (Z)-3-hexenol; 1 octen-3-ol; mixture of 3,4,5,6,6-pentamethyl-3/4-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol;

the aliphatic aldehydes and acetals thereof such as e.g. hexanal; heptanal; octanal; nonanal; decanal; undecanal; dodecanal; tridecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-9-undecenal; 2,6,10 trimethyl-5,9-undecadienal; heptanal diethylacetal; 1,1-dimethoxy-2,2,5 trimethyl-4-hexene; citronellyloxyacetaldehyde; (E/Z)-1-(1-methoxypropoxy)-hex-3-ene; the aliphatic ketones and oximes thereof such as e.g. 2-heptanone; 2-octanone; 3-octanone; 2-nonanone; 5-methyl-3-heptanone; 5-methyl-3 heptanone oxime; 2,4,4,7-tetramethyl-6-octen-3-one; 6-methyl-5-hepten-2-one;

the aliphatic sulfur-containing compounds such as e.g. 3-methylthiohexanol; 3-methylthiohexyl acetate; 3-mercaptohexanol; 3-mercaptohexyl acetate; 3-mercaptohexyl butyrate; 3-acetylthiohexyl acetate; 1-menthene-8-thiol; the aliphatic nitriles such as e.g. 2-nonenenitrile; 2-undecenenitrile; 2 tridecenenitrile; 3,12-tridecadienenitrile; 3,7-dimethyl-2,6-octadienenitrile; 3,7-dimethyl-6 octenenitrile; the esters of aliphatic carboxylic acids such as e.g. (E) and (Z)-3-hexenyl formate; ethyl acetoacetate; isoamyl acetate; hexyl acetate; 3,5,5-trimethylhexyl acetate; 3 methyl-2-butenyl acetate; (E)-2-hexenyl acetate; (E) and (Z)-3-hexenyl acetate; octyl acetate; 3-octyl acetate; 1-octen-3-yl acetate; ethyl butyrate; butyl butyrate; isoamyl butyrate; hexyl butyrate; (E) and (Z)-3-hexenyl isobutyrate; hexyl crotonate; ethyl isovalerate; ethyl 2-methylpentanoate; ethyl hexanoate; allyl hexanoate; ethyl heptanoate; allyl heptanoate; ethyl octanoate; ethyl (E,Z)-2,4-decadienoate; methyl 2-octinate; methyl 2-noninate; allyl 2-isoamyloxy acetate; methyl-3,7-dimethyl-2,6-octadienoate; 4-methyl-2-pentyl crotonate;

the acyclic terpene alcohols such as e.g. geraniol; nerol; linalool; lavandulol; nerolidol; farnesol; tetrahydrolinalool; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2 ol; 3,7-dimethyl-4,6-octadien-3-ol; 3,7-dimethyl-1,5,7-octatrien-3-ol; 2,6-dimethyl-2,5,7-octatrien-1-ol; and the formates, acetates, propionates, isobutyrates, butyrates, isovalerates, pentanoates, hexanoates, crotonates, tiglinates and 3-methyl-2 butenoates thereof;

the acyclic terpene aldehydes and ketones such as e.g. geranial; neral; citronellal; 7 hydroxy-3,7-dimethyloctanal; 7 methoxy-3,7-dimethyloctanal; 2,6,10-trimethyl-9 undecenal; geranyl acetone; as well as the dimethyl and diethylacetals of geranial, neral, 7-hydroxy-3,7-dimethyloctanal; the cyclic terpene alcohols such as e.g. menthol; isopulegol; alpha-terpineol; terpine-4-ol; menthan-8-ol; menthan-1-ol; menthan-7-ol; borneol; isoborneol; linalool oxide; nopol; cedrol; ambrinol; vetiverol; guajol; and the formates, acetates, propionates, isobutyrates, butyrates, isovalerates, pentanoates, hexanoates, crotonates, tiglinates and 3-methyl-2-butenoates thereof;

the cyclic terpene aldehydes and ketones such as e.g. menthone; isomenthone; 8 mercaptomenthan-3-one; carvone; camphor; fenchone; alpha-ionone; beta-ionone; alpha-n-methylionone; beta-n-methylionone; alpha-isomethylionone; beta-isomethylionone; alpha-irone; alpha-damascone; beta-damascone; beta-damascenone; delta-damascone; gamma-damascone; 1-(2,4,4-trimethyl-2-cyclohexen-1-yl)-2-buten-1-one; 1,3,4,6,7,8a-hexahydro-1,1,5,5-tetramethyl-2H-2,4a-methano-naphthalene-8 (5H)-one; 2-methyl-4-(2,6,6-trimethyl-1-cyclohexen-1-yl)-2-butenal; nootkatone; dihydronootkatone; 4,6,8-megastigmatrien-3-one; alpha-sinensal; beta-sinensal; acetylated cedar wood oil (methyl cedryl ketone);

the cyclic alcohols such as e.g. 4-tert-butylcyclohexanol; 3,3,5-trimethylcyclohexanol; 3-isocamphylcyclohexanol; 2,6,9-trimethyl-Z2,Z5,E9-cyclododecatrien-1-ol; 2-isobutyl-4-methyltetrahydro-2H-pyran-4-ol;

the cycloaliphatic alcohols such as e.g. alpha-3,3-trimethylcyclohexylmethanol; 1 (4-isopropylcyclohexyl) ethanol; 2-methyl-4-(2,2,3-trimethyl-3-cyclopent-1-yl) butanol; 2-methyl-4-(2,2,3 trimethyl-3-cyclopent-1-yl)-2-buten-1-ol; 2-ethyl-4-(2,2,3-trimethyl-3 cyclopent-1-yl)-2-buten-1-ol; 3-methyl-5-(2,2,3 trimethyl-3-cyclopent-1-yl) pentan-2 ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl) pentan-3-ol; 1-(2,2,6-trimethylcyclohexyl) hexan-3-ol;

the cyclic and cycloaliphatic ethers such as e.g. cineol; cedryl methyl ether; cyclododecyl methyl ether; 1,1-dimethoxycyclododecane; (ethoxymethoxy)cyclo-dodecane; alpha-cedrene epoxide; 3a,6,6,9a-tetramethyldodecahydronaphtho[2,1-b]furan; 3a-ethyl-6,6,9a-trimethyldodecahydro-naphtho[2,1-b]furan; 1,5,9-trimethyl-13-oxabicyclo-[10.1.0]trideca-4,8-diene; rose oxide; 2-(2,4-dimethyl-3-cyclohexen-1-yl)-5-methyl-5-(1-methylpropyl)-1,3-dioxane;

the cyclic and macrocyclic ketones such as e.g. 4-tert-butylcyclohexanone; 2,2,5 trimethyl-5-pentylcyclopentanone; 2-heptylcyclopentanone; 2-pentylcyclo-pentanone; 2-hydroxy-3-methyl-2-cyclopenten-1-one; 3-methyl-cis-2-penten-1-yl-2 cyclopenten-1-one; 3-methyl-2-pentyl-2-cyclopenten-1-one; 3-methyl-4-cyclopenta-decenone; 3-methyl-5-cyclopentadecenone; 3-methylcyclopentadecanone; 4-(1-ethoxyvinyl)-3,3,5,5-tetramethylcyclohexanone; 4-tert-pentylcyclohexanone; 5-cyclohexadecen-1-one; 6,7-dihydro-1,1,2,3,3-pentamethyl-4 (5H)-indanone; 8-cyclo-hexadecen-1-one; 7-cyclohexadecen-1-one; (7/8)-cyclohexadecen-1-one; 9 cyclo-heptadecen-1-one; cyclopentadecanone; cyclohexadecanone;

the cycloaliphatic aldehydes such as e.g. 2,4-dimethyl-3-cyclohexenecarbaldehyde; 2 methyl-4-(2,2,6-trimethylcyclohexen-1-yl)-2-butenal; 4-(4-hydroxy-4-methylpentyl)-3 cyclohexene carbaldehyde; 4-(4-methyl-3-penten-1-yl)-3-cyclohexenecarbaldehyde;

the cycloaliphatic ketones such as e.g. 1-(3,3-dimethylcyclohexyl)-4-penten-1-one; 2,2 dimethyl-1-(2,4-dimethyl-3-cyclohexen-1-yl)-1-propanone; 1-(5,5-dimethyl-1 cyclo-hexen-1-yl)-4-penten-1-one; 2,3,8,8-tetramethyl-1,2,3,4,5,6,7,8-octahydro-2-naphthalenyl methyl ketone; methyl 2,6,10-trimethyl-2,5,9-cyclododecatrienyl ketone; tert-butyl (2,4-dimethyl-3-cyclohexen-1-yl) ketone;

the esters of cyclic alcohols such as e.g. 2-tert-butylcyclohexyl acetate; 4-tert-butylcyclohexyl acetate; 2-tert-pentylcyclohexyl acetate; 4-tert-pentylcyclohexyl acetate; 3,3,5-trimethylcyclohexyl acetate; decahydro-2-naphthyl acetate; 2-cyclopentylcyclopentyl crotonate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5 or 6-indenyl acetate; 4,7-methano-3a,4,5,6,7,7a-hexahydro-5 or 6 indenyl propionate; 4,7-methano-3a,4,5,6,7,7a-hexahydro-5 or 6-indenyl isobutyrate; 4,7 methanooctahydro-5 or 6-indenyl acetate;

the esters of cycloaliphatic alcohols such as e.g. 1-cyclohexylethyl crotonate;

the esters of cycloaliphatic carboxylic acids such as e.g. allyl 3-cyclohexylpropionate; allyl cyclohexyloxyacetate; cis and trans-methyl dihydrojasmonate; cis and trans-methyl jasmonate; methyl 2-hexyl-3-oxocyclopentanecarboxylate; ethyl 2-ethyl-6,6 dimethyl-2-cyclohexenecarboxylate; ethyl 2,3,6,6-tetramethyl-2 cyclohexene-carboxylate; ethyl 2-methyl-1,3-dioxolane-2-acetate;

the araliphatic alcohols such as e.g. benzyl alcohol; 1-phenylethyl alcohol, 2 phenylethyl alcohol, 3-phenylpropanol; 2-phenylpropanol; 2-phenoxyethanol; 2,2-dimethyl-3-phenylpropanol; 2,2-dimethyl-3-(3-methylphenyl) propanol; 1,1-dimethyl-2 phenylethyl alcohol; 1,1-dimethyl-3-phenylpropanol; 1-ethyl-1-methyl-3-phenylpropanol; 2-methyl-5-phenylpentanol; 3-methyl-5-phenylpentanol; 3-phenyl-2-propen-1-ol; 4-methoxy-benzyl alcohol; 1-(4-isopropylphenyl) ethanol;

the esters of araliphatic alcohols and aliphatic carboxylic acids such as e.g. benzyl acetate; benzyl propionate; benzyl isobutyrate; benzyl isovalerate; 2-phenylethyl acetate; 2-phenylethyl propionate; 2-phenylethyl isobutyrate; 2 phenylethyl isovalerate; 1 phenylethyl acetate; alpha-trichloromethylbenzyl acetate; alpha,alpha-dimethylphenylethyl acetate;

alpha,alpha-dimethylphenylethyl butyrate; cinnamyl acetate; 2-phenoxyethyl isobutyrate; 4-methoxybenzyl acetate;

the araliphatic ethers such as e.g. 2-phenylethyl methyl ether; 2 phenylethyl isoamyl ether; 2-phenylethyl 1-ethoxyethyl ether; phenylacetaldehyde dimethyl acetal; phenylacetaldehyde diethyl acetal; hydratropaaldehyde dimethyl acetal; phenylacetaldehyde glycerol acetal; 2,4,6-trimethyl-4-phenyl-1,3-dioxane; 4,4a,5,9b-tetrahydroindeno[1,2-d]-m-dioxine; 4,4a,5,9b-tetrahydro-2,4-dimethylindeno[1,2-d]-m dioxine;

the aromatic and araliphatic aldehydes such as e.g. benzaldehyde; phenylacetaldehyde; 3-phenylpropanal; hydratropaaldehyde; 4-methylbenzaldehyde; 4 methylphenylacetaldehyde; 3-(4-ethylphenyl)-2,2-dimethylpropanal; 2-methyl-3-(4-isopropylphenyl) propanal; 2-methyl-3-(4-tert-butylphenyl) propanal; 2-methyl-3-(4-isobutylphenyl) propanal; 3-(4-tert-butylphenyl) propanal; cinnamaldehyde; alpha-butylcinnamaldehyde; alpha-amylcinnamaldehyde; alpha-hexylcinnamaldehyde; 3 methyl-5-phenylpentanal; 4-methoxybenzaldehyde; 4-hydroxy-3 methoxy-benzaldehyde; 4-hydroxy-3-ethoxybenzaldehyde; 3,4-methylenedioxybenzaldehyde; 3,4-dimethoxybenzaldehyde; 2-methyl-3-(4-methoxyphenyl) propanal; 2-methyl-3-(4-methylenedioxyphenyl) propanal;

the aromatic and araliphatic ketones such as e.g. acetophenone; 4-methylacetophenone; 4-methoxyacetophenone; 4-tert-butyl-2,6-dimethylaceto-phenone; 4-phenyl-2-butanone; 4-(4-hydroxyphenyl)-2-butanone; 1-(2-naphthalenyl)-ethanone; 2-benzofuranylethanone; (3-methyl-2-benzofuranyl) ethanone; benzophenone; 1,1,2,3,3,6-hexamethyl-5-indanyl methyl ketone; 6-tert-butyl-1,1 dimethyl-4 indanyl methyl ketone; 1-[2,3-dihydro-1,1,2,6-tetramethyl-3-(1-methylethyl)-1H-5 indenyl]ethanone; 5′,6′,7′,8′-Tetrahydro-3′,5′,5′,6′,8′,8′-hexamethyl-2′-acetonaphthone;

the aromatic and aliphatic carboxylic acids and esters thereof such as e.g. benzoic acid; phenylacetic acid; methyl benzoate; ethyl benzoate; hexyl benzoate; benzyl benzoate; methyl phenylacetate; ethyl phenylacetate; geranyl phenylacetate; phenylethyl phenylacetate; methyl cinnamate; ethyl cinnamate; benzyl cinnamate; phenylethyl cinnamate; cinnamyl cinnamate; allyl phenoxyacetate; methyl salicylate; isoamyl salicylate; hexyl salicylate; cyclohexyl salicylate; cis-3-hexenyl salicylate; benzyl salicylate; phenylethyl salicylate; methyl 2,4-dihydroxy-3,6-dimethylbenzoate; ethyl 3-phenylglycidate; ethyl 3-methyl-3-phenylglycidate;

the nitrogen-containing aromatic compounds such as e.g. 2,4,6-trinitro-1,3-dimethyl-5 tert-butylbenzene; 3,5-dinitro-2,6-dimethyl-4-tert-butylacetophenone; cinnamonitrile; 3 methyl-5-phenyl-2-pentenonitrile; 3-methyl-5-phenylpentanonitrile; methyl anthranilate; methyl-N-methylanthranilate; Schiff bases of methyl anthranilate with 7 hydroxy-3,7-dimethyloctanal, 2-methyl-3-(4-tert-butylphenyl) propanal or 2,4 dimethyl-3-cyclohexenecarbaldehyde; 6-isopropylquinoline; 6-isobutylquinoline; 6-sec-butylquinoline; 2-(3-phenylpropyl)pyridine; indole; skatole; 2-methoxy-3 isopropyl-pyrazine; 2-isobutyl-3-methoxypyrazine;

the phenols, phenyl ethers and phenyl esters such as e.g. estragole; anethole; eugenol; eugenyl methyl ether; isoeugenol; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2 ethoxy-5-(1-propenyl) phenol; p-cresyl phenylacetate;

the heterocyclic compounds such as e.g. 2,5-dimethyl-4-hydroxy-2H-furan-3-one; 2 ethyl-4-hydroxy-5-methyl-2H-furan-3-one; 3-hydroxy-2-methyl-4H-pyran-4-one; 2 ethyl-3-hydroxy-4H-pyran-4-one;

the lactones such as e.g. 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide; 4-methyl-1,4-decanolide; 1,15-pentadecanolide; cis and trans-11-pentadecen-1,15-olide; cis and trans-12-pentadecen-1,15-olide; 1,16-hexadecanolide; 9-hexadecen-1,16-olide; 10-oxa-1,16-hexadecanolide; 11-oxa-1,16-hexadecanolide; 12-oxa-1,16-hexadecanolide; ethylene 1,12-dodecanedioate; ethylene 1,13-tridecanedioate; coumarin; 2,3-dihydrocoumarin; octahydrocoumarin.

The aroma chemical (X) used in the composition are obtained from known commercial sources and procured from Germany.

In a preferred embodiment, the composition comprises compound of formula (I) or mixture comprising compound of formula (I) and compound of formula (II) and at least one aroma chemical (X), where the at least one aroma chemical (X) different from the compounds of formula (I) and (II) is preferably selected from the group consisting of: geranyl acetate, alpha-hexylcinnamaldehyde, 2 phenoxyethyl isobutyrate, dihydromyrcenol, methyl dihydrojasmonate (preferably with a content of cis isomer of more than 60 wt. %), 4,6,6,7,8,8 hexamethyl-1,3,4,6,7,8-hexa-hydro-cyclopenta [g]-benzopyran, linalool, tetrahydrolinalool, ethyllinalool, benzyl salicylate, 2 methyl-3-(4-tert-butylphenyl) propanal, cinnamyl alcohol, 4,7-methano-3a,4,5,6,7,7a-hexahydro-5 indenyl acetate, 4,7-methano-3a,4,5,6,7,7a-hexahydro-6-indenyl acetate, cit-ronellol, citronellyl acetate, citronellol, geraniol, tetrahydrogeraniol, vanillin, linalyl acetate, styrolyl acetate, octahydro-2,3,8,8-tetramethyl-2-acetonaphthone, 2-acetyl-1,2,3,4,6,7,8-octahydro-2,3,8,8-tetramethyl-naphthalene, hexyl salicylate, 4-tert-butylcyclohexyl acetate, 2-tert-butylcyclo-hexyl acetate, alpha-ionone, n-alpha-methylionone, alpha-iso-methylionone, coumarin, terpinyl acetate, 2-phenylethyl alcohol, 4-(4-hydroxy-4-methylpentyl)-3-cyclohexenecarboxaldehyde, alpha-amylcinnamaldehyde, ethylene brassylate, (E)-3-methylcyclopentadec-5-enone, (Z)-3-methylcyclo-penta-dec-5-enone, 15-pentadec-11-enolide, 15-pentadec-12-enolide, 15-cyclo-pentadecanolide, 1-(5,6,7,8-tetrahydro-3,5,5,6,8,8-hexamethyl-2-naphthalenyl) ethanone, 2-isobutyl-4-methyltetrahydro-2H pyran-4-ol, 2-ethyl-4-(2,2,3-trimethyl-3-cyclopenten-1-yl)-2-buten-1-ol, cis-3-hexenyl acetate, trans-3-hexenyl acetate, trans-2/cis-6-nonadienol, 2,4-dimethyl-3-cyclohexenecarbox-alde hyde, 2,4,4,7-tetramethyloct-6-en-3-one, 2,6-dimethyl-5-hepten-1-al, borneol, 3-(3-isopropylphenyl) butanal, citral, citronellal, 2-methyl-3-(3,4-methylenedioxyphenyl)-propanal, 3-(4-ethylphenyl)-2,2-dimethylpropanal, 7-methyl-2H-1,5-benzodioxepin-3 (4H)-one, 3,3,5-trimethylcyclohexyl acetate (preferably with a content of cis isomers of 70 wt. % or more), 2,5,5 trimethyl-1,2,3,4,4a,5,6,7-octahydronaphthalen-2-ol, 3-(4-tert-butylphenyl)-propanal, ethyl 2-methylpentanoate, ethoxymethoxycyclododecane, 2,4-dimethyl-4,4a,5,9b-tetrahydroindeno[1,2-d][1,3]dioxine, (2-tert-butyl-cyclo-hexyl)acetate, 3-[5,5,6-trimethylbicyclo[2.2.1]hept-2-yl]cyclohexan-1-ol, menthone, isomenthone, carvone, camphor, beta-ionone, beta-n-methylionone, beta-isomethylionone, alpha-irone, alpha-damascone, beta-damascone, beta-damascenone, delta-damascone, acetylated cedar wood oil, and mixtures thereof.

Non-aroma chemical carrier:

The non-aroma chemical carrier in the composition of the invention is preferably selected from the group consisting of surfactants, oil components antioxidants, deodorant-active agents and solvents.

Preferably the at least one non-aroma chemical carrier is a compound, a mixture of compounds or other additives, which has/have no or no noteworthy sensory properties. The non-aroma chemical carrier can serve for the dilution and/or the fixing of compound of formula (I) or mixture comprising compound of formula (I) and compound of formula (II) and—optionally the at least one aroma chemical (X), as defined above, if comprised in the composition.

The non-aroma chemical carrier in the composition of the invention is preferably selected from surfactants, oil components, solvents, or any mixture of two or more of the aforementioned.

### Solvent:

In the context of the present invention, a “solvent” serves for the dilution of compound of formula (I) or mixture comprising compound of formula (I) and compound of formula (II) to be used according to the invention, without having its own aroma.

The amount of solvent(s) is adjusted depending on the composition and represents a routine task of the person skilled in the art.

Preferably, the solvent is present in the composition in a total amount of 0.01 wt. % to 99.0 wt. %, more preferably in a total amount of 0.05 wt. % to 95.0 wt. %, yet more preferably in a total amount of 0.1 wt. % to 80.0 wt. %, most preferably 0.1 wt. % to 70.0 wt. %, particularly in a total amount of 0.1 wt. % to 60.0 wt. %, based on the total weight of the composition.

In a preferred embodiment, the composition comprises 0.05 wt. % to 10 wt. %, more preferably 0.1 wt. % to 5 wt. %, yet more preferably 0.2 wt. % to 3 wt. % total solvent(s), based on the total weight of the composition. In yet another preferred embodiment of the invention, the composition comprises 20 wt. % to 70 wt. %, more preferably 25 wt. % to 50 wt. % of total solvent(s), based on the total weight of the composition.

Preferred solvents are selected from ethanol, isopropanol, diethylene glycol monoethyl ether, glycerol, propylene glycol, 1,2 butylene glycol, dipropylene glycol, triethyl citrate, isopropyl myristate, or any mixture of two or more of the aforementioned.

In a preferred embodiment, the composition comprises compound of formula (I) or mixture comprising compound of formula (I) and compound of formula (II) and at least one solvent and optionally at least one aroma chemical (X).

### Oil Component:

Preferably, the total oil components are present in an amount of 0.1 to 80 wt. %, more preferably 0.5 to 70 wt. %, yet more preferably 1 to 60 wt. %, even more preferably 1 to 50 wt. %, particularly 1 to 40 wt. %, more particularly 5 to 25 wt. % and specifically 5 to 15 wt. %, based on the total weight of the composition.

Preferably the oil components are selected from Guerbet alcohols based on fatty alcohols containing 6 to 18, preferably 8 to 10, carbon atoms and other additional esters, such as myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of C18-C38 alkyl-hydroxycarboxylic acids with linear or branched C6-C22 fatty alcohols, more especially dioctyl malate, esters of linear and/or branched fatty acids with polyhydric alcohols (for example propylene glycol, dimer dial or trimer triol), triglycerides based on C6-C10 fatty acids, liquid mono-, di- and triglyceride mixtures based on C6-C18 fatty acids, esters of C6-C22 fatty alcohols and/or Guerbet alcohols with aromatic carboxylic acids, more particularly benzoic acid, esters of dicarboxylic acids with polyols containing 2 to 10 car-bon atoms and 2 to 6 hydroxyl groups, vegetable oils, branched primary alcohols, substituted cyclohexanes, linear and branched C6-C22 fatty alcohol carbonates such as, for example, dicaprylyl carbonate (Cetiol® CC), Guerbet carbonates based on fatty alcohols containing 6 to 18, preferably 8 to 10, carbon atoms, esters of benzoic acid with linear and/or branched C6 to C22 alcohols (for example Finsolv® TN), linear or branched, symmetrical or nonsymmetrical dialkyl ethers containing 6 to 22 carbon atoms per alkyl group such as, for example, dicaprylyl ether (Cetiol® OE), ring opening products of epoxidized fatty acid esters with polyols and hydrocarbons, or mixtures thereof.

In a preferred embodiment, the composition comprises compound of formula (I) or mixture comprising compound of formula (I) and compound of formula (II) and at least one oil component and optionally at least one aroma chemical (X).

### Antioxidants:

It is to be understood that antioxidants are able to inhibit or prevent the undesired changes in the compositions to be protected caused by oxygen effects and other oxidative processes. The effect of the antioxidants consists in most cases in them acting as free-radical scavengers for the free radicals which arise during autoxidation.

In a preferred embodiment, the antioxidant is selected from.
amino acids (for example glycine, alanine, arginine, serine, threonine, histidine, tyrosine, tryptophan) and derivatives thereof,
imidazoles (e.g. urocanic acid) and derivatives thereof,
peptides, such as D,L-carnosine, D-carnosine, L-carnosine (=8-Alanyl-L-histidine) and derivatives thereof,
carotenoids, carotenes (e.g. alpha-carotene, beta-carotene, lycopene, lutein) or derivatives thereof,
chlorogenic acid and derivatives thereof,
lipoic acid and derivatives thereof (for example dihydrolipoic acid),
auro-thioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, gamma-linoleyl, cholesteryl and glyceryl esters thereof) and salts thereof,
dilauryl thiodipropionate, distearyl thiodipropionate, thiodipropionic acid and derivatives thereof (esters, ethers, peptides, lipids, nucleotides, nucleosides and salts),
sulfoximine compounds (for example buthionine sulfoximines, homocysteine sulfoximine, buthionine sulfones, penta-, hexa-, heptathionine sulfoximine),
(metal) chelating agents (e.g. alpha-hydroxy fatty acids, palmitic acid, phytic acid, lactoferrin),
alpha-hydroxy acids (for example citric acid, lactic acid, malic acid),
humic acid, bile acid, bile extracts, bilirubin, biliverdin, boldin (=alkaloid from the plant Peumus boldus, boldo extract,
EDTA, EGTA and derivatives thereof,
unsaturated fatty acids and derivatives thereof (e.g. gamma-linolenic acid, linoleic acid, oleic acid),
folic acid and derivatives thereof,
ubiquinone and ubiquinol and derivatives thereof,
vitamin C and derivatives (for example ascorbyl palmitate, Mg ascorbyl phosphate, ascorbyl acetate),
tocopherols and derivatives (for example vitamin E acetate),
vitamin A and derivatives (for example vitamin A palmitate),
coniferyl benzoate of gum benzoin, rutic acid and derivatives thereof, alpha-glycosylrutin, ferulic acid, furfurylideneglucitol,
butylhydroxytoluene (BHT), butylhydroxyanisole (BHA),
nordihydroguaiacic acid, nordihydroguaiaretic acid, trihydroxybutyrophenone, uric acid and derivatives thereof, mannose and derivatives thereof,
superoxide dismutase,
zinc and derivatives thereof (for example ZnO, ZnSO₄),
selenium and derivatives thereof (for example selenomethionine),
stilbenes and derivatives thereof (e.g. stilbene oxide, trans-stilbene oxide),
   or mixtures of two or more of the aforementioned.

In a preferred embodiment, the antioxidant is selected from pentaerythrityl, tetra-di-t-butyl-hydroxyhydrocinnamate, nordihydroguaiaretic acid, ferulic acid, resveratrol, propyl gallate, butylhydroxytoluene (BHT), butylhydroxyanisole (BHA), ascorbyl palmitate, tocopherol, or mixtures of two or more of the aforementioned.

Preferably, the compositions according to the present invention comprise the antioxidant in a total amount of 0.001 to 25 wt.-%, preferably 0.005 to 10 wt.-%, more preferably 0.01 to 8 wt.-%, yet more preferably 0.025 to 7 wt.-%, even more preferably 0.05 to 5 wt.-%, based on the total weight of the composition.

In a preferred embodiment, the composition comprises compound of formula (1) or mixture comprising compound of formula (I) and compound of formula (II) and at least one antioxidant and optionally at least one aroma chemical (X).

### Deodorant-Active Agents

Deodorizing compositions (deodorants and antiperspirants) counteract, mask or eliminate body odors. Body odors are formed through the action of skin bacteria on apocrine perspiration which results in the formation of unpleasant-smelling degradation products.

Preferably the deodorant-active agent is selected from anti-perspirants, esterase inhibitors, antibacterial agents, or mixtures of two or more of the aforementioned.

Suitable antiperspirants are selected from salts of aluminum, zirconium or zinc. Examples are aluminum chloride, aluminum chlorohydrate, aluminum dichlorohydrate, aluminum sesquichlorohydrate and complex compounds thereof, for example with 1,2-propylene glycol, aluminum hydroxyallantoinate, aluminum chloride tartrate, aluminum zirconium trichlorohydrate, aluminum zirconium tetrachlorohydrate, zirconium aluminum pentachlorohydrate and complex compounds thereof, for example with amino acids, such as glycine. Aluminum chlorohydrate, aluminum zirconium tetrachlorohydrate, aluminum zirconium pentachlorohydrate, or complex compounds thereof are preferably used.

Preferably, the anti-perspirant is selected from aluminum chloride, aluminum chlorohydrate, aluminum dichlorohydrate, aluminum sesquichlorohydrate, aluminum hydroxyallantoinate, aluminum chloride tartrate, aluminum zirconium trichlorohydrate, aluminum zirconium tetrachlorohydrate aluminum zirconium pentachlorohydrate, ormixtures of two or more of the aforementioned.

Where perspiration is present in the underarm region, extracellular enzymes-esterases, mainly proteases and/or lipases are formed by bacteria and split the esters present in the perspiration, releasing odors in the process. Suitable esterase inhibitors are for example trialkyl citrates, such as trimethyl citrate, tripropyl citrate, triisopropyl citrate, tributyl citrate and, in particular, triethyl citrate. Esterase inhibitors inhibit enzyme activity and thus reduce odor formation. The free acid is probably released by the cleavage of the citric acid ester and reduces the pH value of the skin to such an extent that the enzymes are inactivated by acylation. Other esterase inhibitors are sterol sulfates or phosphates such as, for example, lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, for example citric acid, malic acid, tartaric acid or tartaric acid diethyl ester, zinc glycinate and mixtures of two or more of the aforementioned.

Preferably, the esterase inhibitor is selected from trimethyl citrate, tripropyl citrate, triisopropyl citrate, tributyl citrate triethyl citrate, lanosterol, cholesterol, campesterol, stigmasterol, sitosterol sulfate, sitosterol phosphate, glutaric acid, glutaric acid monoethyl ester, glutaric acid diethyl ester, adipic acid, adipic acid monoethyl ester, adipic acid diethyl ester, malonic acid, malonic acid diethyl ester, citric acid, malic acid, tartaric acid, tartaric acid diethyl ester zinc glycinate, or mixtures of two or more of the aforementioned.

Preferably, the compositions according to the present invention comprise the esterase inhibitor in a total amount in the range of 0.01 to 20 wt.-%, preferably 0.1 to 10 wt.-% and more particularly 0.5 to 5 wt.-%, based on the total weight of the composition.

The term “anti-bacterial agents” as used herein encompasses substances which have bactericidal and/or bacteriostatic properties. Typically these substances act against gram-positive bacteria such as, for example, 4-hydroxybenzoic acid and salts and esters thereof, N-(4-chlorophenyl)-N′-(3,4-dichlorophenyl)-urea, 2,4,4′-trichloro-2′-hydroxydiphenylether (triclosan), 4-chloro-3,5-dimethylphenol, 2,2′-methylene-bis-(6-bromo-4-chlorophenol), 3-methyl-4-(1-methylethyl)-phenol, 2-benzyl-4-chlorophenol, 3-(4-chlorophenoxy)-propane-1,2-diol, 3-iodo-2-propinyl butyl carbamate, chlorhexidine, 3,4,4′-trichlorocarbanilide (TTC), phenoxyethanol, glycerol monocaprate, glycerol monocaprylate, glycerol monolaurate (GML), diglycerol monocaprate (DMC), salicylic acid-N-alkylamides such as, for example, salicylic acid-n-octyl amide or salicylic acid-n-decyl amide.

Preferably, the antibacterial agent is selected from chitosan, phenoxyethanol, 5-chloro-2-(2,4-dichlorophenoxy)-phenol, 4-hydroxybenzoic acid and salts and esters thereof, N-(4-chlorophenyl)-N′-(3,4-dichlorophenyl)-urea, 2,4,4′-trichloro-2′-hydroxydiphenylether (triclosan), 4-chloro-3,5-dimethylphenol, 2,2′-methylene-bis-(6-bromo-4-chlorophenol), 3-methyl-4-(1-methylethyl)-phenol, 2-benzyl-4-chlorophenol, 3-(4-chlorophenoxy)-propane-1,2-diol, 3-iodo-2-propinyl butyl carbamate, chlorhexidine, 3,4,4′-trichlorocarbanilide (TTC), phenoxyethanol, glycerol monocaprate, glycerol monocaprylate, glycerol monolaurate (GML), diglycerol monocaprate (DMC), salicylic acid-N-alkylamides, or mixtures of two or more of the aforementioned.

Preferably, the composition according to the present invention comprises the antibacterial agent(s) in a total amount in the range of 0.01 to 5 wt. % and preferably 0.1 to 2 wt.-%, based on the total weight of the composition.

In a preferred embodiment, the composition comprises compound of formula (1) or mixture comprising compound of formula (I) and compound of formula (II) and at least one deodorant active agent and optionally at least one aroma chemical (X).

### Surfactants

Preferably, the surfactant is selected from anionic, non-ionic, cationic, amphoteric, zwitterionic surfactant, or a mixture of two or more of the aforementioned. More preferably, the surfactant is an anionic surfactant.

Preferably, the compositions according to the invention contain the surfactant(s), in a total amount of 0 to 40 wt. %, more preferably 0 to 20 wt. %, more preferably 0.1 to 15 wt. %, and particularly 0.1 to 10 wt. %, based on the total weight of the composition.

Preferable nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides or glucuronic acid derivatives, fatty acid-N-alkyl glucamides, protein hydrolysates (particularly wheat-based vegetable products), polyol fatty acid esters, sugar esters, sorbitan esters, polysorbates and amine oxides. If the nonionic surfactants contain polyglycol ether chains, they may have a conventional homolog distribution, although they preferably have a narrow-range homolog distribution.

Zwitterionic surfactants are surface-active compounds which contain at least one quaternary ammonium group and at least one COO (−) or SO₃ (−) group in the molecule.

Particularly suitable zwitterionic surfactants are the so-called betaines, such as the N-alkyl-N,N-dimethyl ammonium glycinates, for example, cocoalkyl dimethyl ammonium glycinate, N-acylaminopropyl-N,N-dimethyl ammonium glycinates, for example, cocoacylaminopropyl dimethyl ammonium glycinate, and 2-alkyl-3-carboxymethyl-3-hydroxyethyl imidazolines, containing 8 to 18 carbon atoms in the alkyl or acyl group, and cocoacylaminoethyl hydroxyethyl carboxymethyl glycinate. The fatty acid amide derivative known under the CTFA name of Cocamidopropyl Betaine is particularly preferred.

Ampholytic surfactants are also suitable, particularly as co-surfactants. Ampholytic surfactants are surface-active compounds which, in addition to a C8 to C18 alkyl or acyl group, contain at least one free amino group and at least one —COOH or —SO₃H group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-cocoalkylaminopropionate, cocoacylaminoethyl aminopropionate and acyl sarcosine.

Anionic surfactants are characterized by a water-solubilizing anionic group such as, for example, a carboxylate, sulfate, sulfonate or phosphate group and a lipophilic group. Dermatologically safe anionic surfactants are known to the practitioner in large numbers from relevant textbooks and are commercially available. They are, in particular, alkyl sulfates in the form of their alkali metal, ammonium or alkanolammonium salts, alkylether sulfates, alkylether carboxylates, acyl isethionates, acyl sarcosinates, acyl taurines containing linear C₁₂-C₁₈ alkyl or acyl groups and sulfosuccinates and acyl glutamates in the form of their alkali metal or ammonium salts.

Particularly suitable cationic surfactants are quaternary ammonium compounds, preferably ammonium halides, more especially chlorides and bromides, such as alkyl trimethyl ammonium chlorides, dialkyl dimethyl ammonium chlorides and trialkyl methyl ammonium chlorides, for example, cetyl trimethyl ammonium chloride, stearyl trim ethyl ammonium chloride, distearyl dimethyl ammonium chloride, lauryl dimethyl ammonium chloride, lauryl dimethyl benzyl ammonium chloride and tricetyl methyl ammonium chloride. In addition, the readily biodegradable quaternary ester compounds, such as, for example, the dialkyl ammonium methosulfates and methyl hydroxyalkyl dialkoyloxyalkyl ammonium methosulfates marketed under the name of Stepantexe and the corresponding products of the Dehyquart® series, may be used as cationic surfactants. “Esterquats” are generally understood to be quaternized fatty acid triethanolamine ester salts. They can provide the compositions with particular softness. They are known substances which are prepared by the relevant methods of organic chemistry. Other cationic surfactants suitable for use in accordance with the invention are the quaternized protein hydrolysates.

Due to the characteristic sensory property of compound of formula (I) and its substantivity, tenacity as well as stability, it can especially be used to provide an odor, preferably a fragrance impression to surfactant-containing compositions such as, for example, cleaners (in particular laundry care products and all-purpose cleaners). It can preferably be used to impart a long-lasting notes selected from fresh note, minty note, moldy note, carrot like note, natural note, rooty note, violet note, powdery note, floral note, woody note, warm note, ionone note, fruity note, camphoraceous note, citrus note, herbal note, ethereal note, gourmand note, anisic note, ambrinol like note, animalic note, civet note, soft note, incense note, musty note, fatty note, tobacco note, green note to a surfactant comprising composition.

In a preferred embodiment, the composition comprises compound of formula (1) or mixture comprising compound of formula (I) and compound of formula (II) and at least one surfactant and optionally at least one aroma chemical (X).

Suitable compositions are for example perfume compositions, body care compositions (including cosmetic compositions and products for oral and dental hygiene), hygiene articles, cleaning compositions (including dishwashing compositions), textile detergent compositions, compositions for scent dispensers, foods, food supplements, pharmaceutical compositions and crop protection compositions.

Perfume compositions can be selected from fine fragrances, air fresheners in liquid form, gel-like form or a form applied to a solid carrier, aerosol sprays, scented cleaners, perfume candles, or oils, such as lamp oils or oils for massage.

Examples for fine fragrances are perfume extracts, Eau de Parfums, Eau de Toilettes, Eau de Colognes, Eau de Solide and Extrait Parfum.

Body care compositions include cosmetic compositions and products for oral and dental hygiene, and can be selected from after-shaves, pre-shave products, splash colognes, solid and liquid soaps, shower gels, shampoos, shaving soaps, shaving foams, bath oils, cosmetic emulsions of the oil-in-water type, of the water-in-oil type and of the water-in-oil-in-water type, such as e.g. skin creams and lotions, face creams and lotions, sunscreen creams and lotions, after-sun creams and lotions, hand creams and lotions, foot creams and lotions, hair removal creams and lotions, after-shave creams and lotions, tanning creams and lotions, hair care products such as e.g. hairsprays, hair gels, setting hair lotions, hair conditioners, hair shampoo, permanent and semi-permanent hair colorants, hair shaping compositions such as cold waves and hair smoothing compositions, hair tonics, hair creams and hair lotions, deodorants and antiperspirants such as e.g. underarm sprays, roll-ons, deodorant sticks and deodorant creams, products of decorative cosmetics such as e.g. eye-liners, eye-shadows, nail varnishes, make-ups, lipsticks and mascara, and products for oral and dental hygiene, such as toothpaste, dental floss, mouth wash, breath fresheners, dental foam, dental gels and dental strips.

Hygiene articles can be selected from joss sticks, insecticides, repellents, propellants, rust removers, perfumed freshening wipes, armpit pads, baby diapers, sanitary towels, toilet paper, cosmetic wipes, pocket tissues, dishwasher and deodorizer.

Cleaning compositions, such as e.g. cleaners for solid surfaces, can be selected from perfumed acidic, alkaline and neutral cleaners, such as e.g. floor cleaners, window cleaners, dishwashing compositions both for handwashing and machine washing use, bath and sanitary cleaners, scouring milk, solid and liquid toilet cleaners, powder and foam carpet cleaners, waxes and polishes such as furniture polishes, floor waxes, shoe creams, disinfectants, surface disinfectants and sanitary cleaners, brake cleaners, pipe cleaners, limescale removers, grill and oven cleaners, algae and moss removers, mold removers, facade cleaners.

Textile detergent compositions can be selected from liquid detergents, powder detergents, laundry pre-treatments such as bleaches, soaking agents and stain removers, fabric softeners, washing soaps, washing tablets.

Food means a raw, cooked, or processed edible substance, ice, beverage or ingredient used or intended for use in whole or in part for human consumption, or chewing gum, gummies, jellies, and confectionaries.

A food supplement is a product intended for ingestion that contains a dietary ingredient intended to add further nutritional value to the diet. A dietary ingredient may be one, or any combination, of the following substances: a vitamin, a mineral, an herb or other botanical, an amino acid, a dietary substance for use by people to supplement the diet by increasing the total dietary intake, a concentrate, metabolite, constituent, or extract. Food supplements may be found in many forms such as tablets, capsules, soft gels, gel caps, liquids, or powders.

Pharmaceutical compositions comprise compositions which are intended for use in the diagnosis, cure, mitigation, treatment, or prevention of disease as well as articles (other than food) intended to affect the structure or any function of the body of man or other animals.

Crop protection compositions comprise compositions which are intended for the managing of plant diseases, weeds and other pests (both vertebrate and invertebrate) that damage agricultural crops and forestry.

Preferably, the compositions according to the invention further comprises at least one auxiliary agent selected from the group consisting of preservatives, abrasives, anti-acne agents, agents to combat skin aging, anti-cellulite agents, antidandruff agents, anti-inflammatory agents, irritation-preventing agents, irritation-alleviating agents, astringents, sweat-inhibiting agents, antiseptics, anti-statics, binders, buffers, carrier materials, chelating agents, cell stimulants, care agents, hair removal agents, emulsifiers, enzymes, essential oils, fibers, film formers, fixatives, foam formers, foam stabilizers, substances for preventing foaming, foam boosters, fungicides, gelling agents, gel-forming agents, hair care agents, hair shaping agents, hair smoothing agents, moisture-donating agents, moisturizing substances, humectant substances, bleaching agents, strengthening agents, stain removal agents, optical brighteners, impregnating agents, soil repellents, friction-reducing agents, lubricants, moisturizing creams, ointments, opacifiers, plasticizers, covering agents, polish, shine agents, polymers, powders, proteins, refatting agents, exfoliating agents, silicones, skin-calming agents, skin-cleansing agents, skin care agents, skin-healing agents, skin lightening agents, skin-protective agents, skin-softening agents, cooling agents, skin-cooling agents, warming agents, skin-warming agents, stabilizers, UV-absorbent agents, UV filters, fabric softeners, suspending agents, skin-tanning agents, thickeners, vitamins, waxes, fats, phospholipids, saturated fatty acids, mono or polyunsaturated fatty acids, alpha hydroxy acids, polyhydroxy fatty acids, liquefiers, dyes, color-protection agents, pigments, anti-corrosives, polyols, electrolytes and silicone derivatives.

Preparation of compositions and methods to impart an aroma impression to a composition:

One embodiment of the present invention is directed to a method for preparing a composition comprising compound of formula (1) or mixture comprising compound of formula (I) and compound of formula (II) and
(i) at least one aroma chemical (X) other than compounds according to the present invention specifically, compounds of formula I, compounds of formula II, including the stereoisomers thereof, or
(ii) at least one non-aroma chemical carrier, or both of (i) and (ii).

For example, the method can be carried out by mixing the compound of formula (I) or mixture comprising compound of formula (I) and compound of formula (II) and:
(i) at least one aroma compound (X) other than compounds according to the present invention, specifically, compounds of formula I, compounds of formula II, including the stereoisomers thereof, or
(ii) at least one non-aroma chemical carrier, or
(iii) both of (i) and (ii).

The invention is also directed to a method for boosting the aroma impression of a composition, wherein the method comprises incorporating compound of formula (I) or mixture comprising compound of formula (I) and compound of formula (II) into a composition.

In particular, the invention is directed to a method of preparing a perfume composition, body care composition, hygiene article, cleaning composition, textile detergent composition, composition for scent dispensers, food, food supplement, pharmaceutical composition or crop protection composition, comprising including compound of formula (I) into a perfume composition, body care composition, hygiene article, cleaning composition, textile detergent composition, composition for scent dispensers, food, food supplement, pharmaceutical composition, or crop protection composition.

In one embodiment the invention is directed to a method for imparting a note reminiscent of a fresh note, minty note, moldy note, carrot like note, natural note, rooty note, violet note, powdery note, floral note, woody note, warm note, lonone note, fruity note, camphoraceous note, citrus note, herbal note, ethereal note, gourmand note, anisic note, ambrinol like note, animalic note, civet note, soft note, incense note, musty note, fatty note, tobacco note, green note, spicy note, agrumic note, juniper note, pepper note, spearmint note, cedarwood note, dandelion note, earthy note, food-like note, or a combination of two or more notes to a perfume composition, body care composition, hygiene article, cleaning composition, textile detergent composition, composition for scent dispensers, food, food supplement, pharmaceutical composition or crop protection composition, which comprises including compound of formula (I) into a perfume composition, body care composition, hygiene article, cleaning composition, textile detergent composition, composition for scent dispensers, food, food supplement, pharmaceutical composition, or crop protection composition.

### Amounts

Generally, the total amount of compounds of compound of formula (I), compound of formula (II) in the compositions, methods and uses according to the present invention is typically adapted to the particular intended use or the intended application and can, thus, vary over a wide range. As a rule, the customary standard commercial amounts for aroma chemicals, preferably for scents are used.

Preferably the compositions according to the invention comprise compounds of formula (I) in a total amount of 0.001 to 99.9 wt. %, based on the total weight of the composition.

Particularly, the compositions comprise compound of formula (I) or mixture with compound of formula (II) in a total amount of 0.001 to 99.5 wt. %, preferably of 50 to 99 wt. %, more preferably of 80 to 95 wt. % and in particular of 90 to 95 wt. %, based on the total weight of the composition.

Particularly, the compositions comprise compounds of compound of formula (1) or its mixture with compound of formula (II) in a total amount of 0.005 to 80 wt. %, preferably of 0.1 to 30 wt. %, more preferably of 1 to 20 wt. %, and in particular of 5 to 15 wt. %, based on the total weight of the composition.

Particularly, the compositions comprise the compounds of compound of formula (I) or its mixture with compound of formula (II) in a total amount of 0.001 to 20 wt. %, preferably of 0.005 to 6 wt. %, more preferably of 0.05 to 4 wt. %, and in particular of 0.1 to 3 wt. %, based on the total weight of the composition.

### Embodiments

In the following, there is provided a list of embodiments to further illustrate the present disclosure without intending to limit the disclosure to the specific embodiments listed below

1. Use of at least one compound of formula (I)

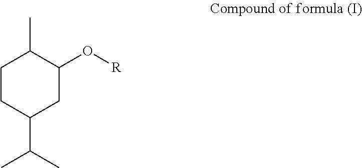

or stereoisomer thereof or a mixture of different stereoisomers thereof,
where,
---- represents a single bond or a double bond,
R is linear or branched C₁-C₆-alkyl, formyl, or linear or branched C₁-C₄-alkylcarbonyl as an aroma chemical; or to impart an aroma impression to a composition.

2. The use according to embodiment 1, wherein the compound of formula (I) is compound of formula (Ia) (i.e. a compound (I) wherein ---- is a single bond),

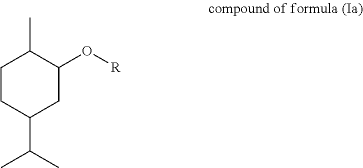

or stereoisomer thereof or a mixture of different stereoisomers thereof,
where
R is linear or branched C₁-C₆-alkyl, formyl, or linear or branched C₁-C₄-alkylcarbonyl.

3. The use according to embodiment 1, wherein the compound of formula (I) is compound of formula (Ib) (i.e. a compound (I) wherein ---- is a double bond)

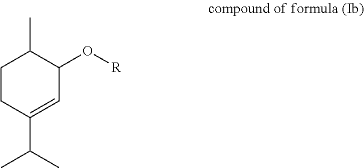

or stereoisomer thereof or a mixture of different stereoisomers thereof,
where
R is linear or branched C₁-C₆ alkyl, formyl, or linear or branched C₁-C₄-alkylcarbonyl.

4. The use according to any of the embodiments 1 to 3, wherein, R is linear or branched C₁-C₆-alkyl, and is preferably selected from methyl, ethyl, propyl, isopropyl, n-butyl, isobutyl, tert-butyl, pentyl, or hexyl.

5. The use according to embodiment 4, wherein R is methyl or ethyl.

6. The use according to any of the embodiments 1 to 3, wherein R is formyl, or linear or branched C₁-C₄ alkylcarbonyl.

7. The use according to embodiment 6, wherein R is linear or branched C₁-C₄-alkylcarbonyl, and is preferably selected from, methyl carbonyl or ethyl carbonyl, n-propyl carbonyl, isopropyl carbonyl, n-butyl carbonyl, isobutyl carbonyl or tert-butyl carbonyl.

8. The use according to embodiments 7, wherein R is linear or branched C₁-C₃-alkylcarbonyl, in particular methylcarbonyl, ethylcarbonyl or isopropylcarbonyl, and specifically methyl carbonyl or ethyl carbonyl.

9. The use according to any of embodiments 1, 2 and 6 to 8, wherein the compound of formula (1) is a compound of formula (Ia), wherein R is linear or branched C₁-C₃-alkylcarbonyl, preferably methylcarbonyl, ethylcarbonyl or isopropylcarbonyl, and more preferably methylcarbonyl or ethylcarbonyl.

10. The use according to any of the preceding embodiments, where the compound of formula (I) is used in admixture with a compound of formula (II)

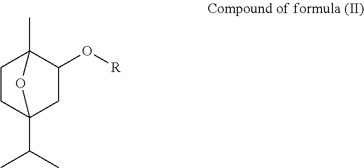

or a stereoisomer thereof or a mixture of different stereoisomers thereof,
where
R is as defined in any of embodiments 1 and 4 to 9; where preferably R has the same meaning in the compounds of formula (I) and (II).

11. The use according to any of the preceding embodiments, as a fragrance or to impart a fragrance to a composition.

12. A method of imparting an aroma impression to a composition comprising at least the step of adding at least one compound of formula (I) according to any of the embodiments 1 to 9 or a mixture according to embodiment 10 to a composition to be aromatized, or mixing at least one compound of formula (I) according to any of embodiments 1 to 9 or a mixture according to embodiment 10 simultaneously or consecutively with the other components of said composition, or mixing at least one compound of formula (I) according to any of embodiments 1 to 9 or a mixture according to embodiment 10 with a pre-formed mixture of a part of the other components of said composition.

13. The use or method according to any of the embodiments 1 to 12, wherein the composition is selected from perfume compositions, body care compositions, hygiene articles, cleaning compositions, textile detergent compositions, compositions for scent dispensers, foods, food supplements, pharmaceutical compositions, or crop protection compositions.

14. The use or method according to any of the embodiments 1 to 13, wherein the aroma impression is selected from fresh note, minty note, moldy note, carrot like note, natural note, rooty note, violet note, powdery note, floral note, woody note, warm note, lonone note, fruity note, camphoraceous note, citrus note, herbal note, ethereal note, gourmand note, anisic note, ambrinol like note, animalic note, civet note, soft note, incense note, musty note, fatty note, tobacco note, green note, spicy note, agrumic note, juniper note, pepper note, spearmint note, cedarwood note, dandelion note, earthy note, food-like note, or a combination of two or more notes.

15. The use or method according to any of the embodiments 1 to 14, wherein the compounds of formula (I) is present in a total amount in the range of ≥0.01 wt.-% to ≤70.0 wt.-%, based on the total weight of the composition.

16. A compound of formula (Ia)

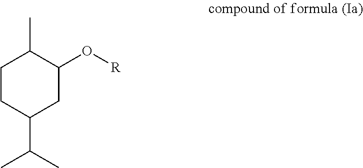

or a stereoisomer thereof or a mixture of different stereoisomers thereof,
wherein R is isopropylcarbonyl.

17. A compound of formula (Ib)

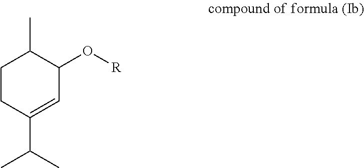

or stereoisomer thereof,
wherein,
R is linear or branched C₁-C₆ alkyl, where R is preferably selected from methyl, ethyl, propyl, isopropyl, n-butyl, isobutyl, tert-butyl, pentyl, or hexyl.

18. The compound of formula (Ib), wherein R is methyl or ethyl, and is preferably ethyl.

19. A composition comprising at least one compound of formula (I) according to any of the embodiments 1 to 9 and
(i) at least one aroma chemical (X) different from the compound of formula (I), or
(ii) at least one non-aroma chemical carrier, or
(iii) both of (i) and (ii).

20. The composition according to embodiment 19, wherein the at least one compound of formula (I) is present in a total amount in the range of ≥0.01 wt.-% to ≤70.0 wt.-%, based on the total weight of the composition.

21. The composition according to any of embodiments 19 or 20, wherein the at least one non-aroma chemical carrier (ii) is selected from surfactants, oil components, antioxidants, deodorant-active agents, or solvents.

22. The composition according to any of the embodiments 19 to 21, wherein the composition is selected from perfume compositions, body care compositions, hygiene articles, cleaning compositions, textile detergent compositions, compositions for scent dispensers, foods, food supplements, pharmaceutical compositions, or crop protection compositions.

23. The composition according to any of the embodiments 19 to 22, comprising at least one compound of formula (I) according to any of the claims 1 to 9, at least one aroma chemical (X) different from the compound of formula (I) and optionally at least one non-aroma chemical carrier, where the at least one aroma chemical (X) different from the compound of formula (I) is selected from the group consisting of: geranyl acetate, alpha-hexylcinnamaldehyde, 2 phenoxyethyl isobutyrate, dihydromyrcenol, methyl dihydrojasmonate (preferably with a content of cis isomer of more than 60 wt. %), 4,6,6,7,8,8 hexamethyl-1,3,4,6,7,8-hexa-hydro-cyclopenta [g]-benzopyran, linalool, tetrahydrolinalool, ethyllinalool, benzyl salicylate, 2 methyl-3-(4-tert-butylphenyl) propanal, cinnamyl alcohol, 4,7-methano-3a,4,5,6,7,7a-hexahydro-5 indenyl acetate, 4,7-methano-3a,4,5,6,7,7a-hexahydro-6-indenyl acetate, cit-ronellol, citronellyl acetate, citronellol, geraniol, tetrahydrogeraniol, vanillin, linalyl acetate, styrolyl acetate, octahydro-2,3,8,8-tetramethyl-2-acetonaphthone, 2-acetyl-1,2,3,4,6,7,8-octahydro-2,3,8,8-tetramethyl-naphthalene, hexyl salicylate, 4-tert-butylcyclohexyl acetate, 2-tert-butylcyclo-hexyl acetate, alpha-ionone, n-alpha-methylionone, alpha-iso-methylionone, coumarin, terpinyl acetate, 2-phenylethyl alcohol, 4-(4-hydroxy-4-methylpentyl)-3-cyclohexenecarboxaldehyde, alpha-amylcinnamaldehyde, ethylene brassylate, (E)-3-methylcyclopentadec-5-enone, (Z)-3-methylcyclo-penta-dec-5-enone, 15-pentadec-11-enolide, 15-pentadec-12-enolide, 15-cyclo-pentadecanolide, 1-(5,6,7,8-tetrahydro-3,5,5,6,8,8-hexamethyl-2-naphthalenyl) ethanone, 2-isobutyl-4-methyltetrahydro-2H pyran-4-ol, 2-ethyl-4-(2,2,3-trimethyl-3-cyclopenten-1-yl)-2-buten-1-ol, cis-3-hexenyl acetate, trans-3-hexenyl acetate, trans-2/cis-6-nonadienol, 2,4-dimethyl-3-cyclohexenecarbox-alde hyde, 2,4,4,7-tetramethyloct-6-en-3-one, 2,6-dimethyl-5-hepten-1-al, borneol, 3-(3-isopropylphenyl) butanal, citral, citronellal, 2-methyl-3-(3,4-methylenedioxyphenyl)-propanal, 3-(4-ethylphenyl)-2,2-dimethylpropanal, 7-methyl-2H-1,5-benzodioxepin-3 (4H)-one, 3,3,5-trimethylcyclohexyl acetate (preferably with a content of cis isomers of 70 wt. % or more), 2,5,5 trimethyl-1,2,3,4,4a,5,6,7-octahydronaphthalen-2-ol, 3-(4-tert-butylphenyl)-propanal, ethyl 2-methylpentanoate, ethoxymethoxycyclododecane, 2,4-dimethyl-4,4a,5,9b-tetrahydroindeno[1,2-d][1,3]dioxine, (2-tert-butyl-cyclo hexyl)acetate, 3-[5,5,6-trimethylbicyclo[2.2.1]hept-2-yl]cyclohexan-1-ol, menthone, isomenthone, carvone, camphor, beta-ionone, beta-n-methylionone, beta-isomethylionone, alpha-irone, alpha-damascone, beta-damascone, beta-damascenone, delta-damascone, acetylated cedar wood oil, and mixtures thereof.

24. The composition according to any of the embodiments 18 to 22, where the non-aroma chemical carriers are selected from the group consisting of surfactants, emollients and solvents, where the solvents are selected from the group consisting of ethanol, isopropanol, propylene glycol, dipropylene glycol, 1,2-butylene glycol, diethylene glycol monoethyl ether, glycerol, diethyl phthalate, isopropyl myristate, triethyl citrate, and benzyl benzoate.

25. A mixture comprising at least one compound of formula (I)

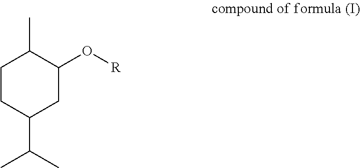

or stereoisomers thereof or a mixture of different stereoisomers thereof,
where
---- represents a single bond or a double bond, and
R is linear or branched C₁-C₆-alkyl, formyl, or linear or branched C₁-C₄-alkylcarbonyl;
and
at least one compound of formula (II)

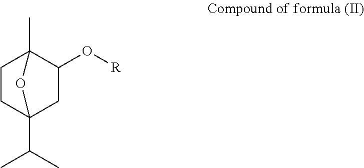

or a stereoisomer thereof or a mixture of different stereoisomers thereof,
where
R is linear or branched C₁-C₆ alkyl, formyl, or linear or branched C₁-C₄-alkylcarbonyl;
where in the compounds of formula (I) and (II) R has preferably the same meaning.

26. The mixture according to embodiment 25, wherein R is linear or branched C₁-C₆-alkyl; where R is preferably selected from methyl, ethyl, propyl, isopropyl, n-butyl, isobutyl, tert-butyl, pentyl, or hexyl.

27. The mixture according to embodiment 26, wherein R is methyl or ethyl.

28. The mixture according to embodiment 25, wherein R is formyl, or linear or branched C₁-C₄ alkylcarbonyl.

29. The mixture according to embodiment 28, wherein R is linear or branched C₁-C₄-alkylcarbonyl; where R is preferably selected from methyl carbonyl, ethyl carbonyl, n-propyl carbonyl, isopropyl carbonyl, n-butyl carbonyl, isobutyl carbonyl, or tert-butyl carbonyl.

30. The mixture according to embodiment 29, wherein R is linear or branched C₁-C₃-alkylcarbonyl; where R is preferably methylcarbonyl, ethylcarbonyl or isopropylcarbonyl, and is more preferably methylcarbonyl or ethylcarbonyl.

31. The mixture according to any of embodiment s25 to 30, wherein the weight ratio of the total amount of the compounds of formula (I) and the total amount of the compounds of formula (II) is in the range 99.99:0.01 to 0.01:99.99, preferably from 100:1 to 1:1, more preferably from 50:1 to 1:1, in particular from 35:1 to 2:1, and more particularly from 25:1 to 3:1, e.g. from 20:1 to 4:1.

32. A composition comprising,
(i) the mixture according to any of the embodiments 25 to 31, and
(ii) at least one aroma chemical (X) other than compounds of formula (I) and (II), or
(iii) at least one non-aroma chemical carrier, or
(iv) both of (ii) and (iii).

33. The composition according to embodiment 32, wherein compounds (I) and (II) are present in a total amount in the range of ≥0.01 wt.-% to ≤70.0 wt.-%, based on the total weight of the composition.

34. The composition according to any of embodiments 32 or 33, wherein at least one non-aroma chemical carrier (ii) is selected from surfactants, oil components, antioxidants, deodorant-active agents, or solvents.

35. The composition according to any of the embodiments 32 to 34, wherein the composition is selected from perfume compositions, body care compositions, hygiene articles, cleaning compositions, textile detergent compositions, compositions for scent dispensers, foods, food supplements, pharmaceutical compositions, or crop protection compositions.

36. The composition according to any of the embodiments 32 to 35, comprising a mixture according to any of embodiments 24 to 30, at least one aroma chemical (X) different from the compounds of formula (I) and (II) and optionally at least one non-aroma chemical carrier, where the at least one aroma chemical (X) different from the compounds of formula (I) and (II) is selected from the group consisting of: geranyl acetate, alpha-hexylcinnamaldehyde, 2 phenoxyethyl isobutyrate, dihydromyrcenol, methyl dihydrojasmonate (preferably with a content of cis isomer of more than 60 wt. %), 4,6,6,7,8,8 hexamethyl-1,3,4,6,7,8-hexa-hydro-cyclopenta [g]-benzopyran, linalool, tetrahydrolinalool, ethyllinalool, benzyl salicylate, 2 methyl-3-(4-tert-butylphenyl) propanal, cinnamyl alcohol, 4,7-methano-3a,4,5,6,7,7a-hexahydro-5 indenyl acetate, 4,7-methano-3a,4,5,6,7,7a-hexahydro-6-indenyl acetate, cit-ronellol, citronellyl acetate, citronellol, geraniol, tetrahydrogeraniol, vanillin, linalyl acetate, styrolyl acetate, octahydro-2,3,8,8-tetramethyl-2-acetonaphthone, 2-acetyl-1,2,3,4,6,7,8-octahydro-2,3,8,8-tetramethyl-naphthalene, hexyl salicylate, 4-tert-butylcyclohexyl acetate, 2-tert-butylcyclo hexyl acetate, alpha-ionone, n-alpha-methylionone, alpha-iso-methylionone, coumarin, terpinyl acetate, 2-phenylethyl alcohol, 4-(4-hydroxy-4-methylpentyl)-3-cyclohexenecarboxaldehyde, alpha-amylcinnamaldehyde, ethylene brassylate, (E)-3-methylcyclopentadec-5-enone, (Z)-3-methylcyclo-penta-dec-5-enone, 15-pentadec-11-enolide, 15-pentadec-12-enolide, 15-cyclo-pentadecanolide, 1-(5,6,7,8-tetrahydro-3,5,5,6,8,8-hexamethyl-2-naphthalenyl) ethanone, 2-isobutyl-4-methyltetrahydro-2H pyran-4-ol, 2-ethyl-4-(2,2,3-trimethyl-3-cyclopenten-1-yl)-2-buten-1-ol, cis-3-hexenyl acetate, trans-3-hexenyl acetate, trans-2/cis-6-nonadienol, 2,4-dimethyl-3-cyclohexenecarbox-alde hyde, 2,4,4,7-tetramethyloct-6-en-3-one, 2,6-dimethyl-5-hepten-1-al, borneol, 3-(3-isopropylphenyl) butanal, citral, citronellal, 2-methyl-3-(3,4-methylenedioxyphenyl)-propanal, 3-(4-ethylphenyl)-2,2-dimethylpropanal, 7-methyl-2H-1,5-benzodioxepin-3 (4H)-one, 3,3,5-trimethylcyclohexyl acetate (preferably with a content of cis isomers of 70 wt. % or more), 2,5,5 trimethyl-1,2,3,4,4a,5,6,7-octahydronaphthalen-2-ol, 3-(4-tert-butylphenyl)-propanal, ethyl 2-methylpentanoate, ethoxymethoxycyclododecane, 2,4-dimethyl-4,4a,5,9b-tetrahydroindeno[1,2-d][1,3]dioxine, (2-tert-butyl-cyclo hexyl)acetate, 3-[5,5,6-trimethylbicyclo[2.2.1]hept-2-yl]cyclohexan-1-ol, menthone, isomenthone, carvone, camphor, beta-ionone, beta-n-methylionone, beta-isomethylionone, alpha-irone, alpha-damascone, beta-damascone, beta-damascenone, delta-damascone, acetylated cedar wood oil, and mixtures thereof.

37. The composition according to any of the embodiments 32 to 36, where the non-aroma chemical carriers are selected from the group consisting of surfactants, emollients and solvents, where the solvents are selected from the group consisting of ethanol, isopropanol, propylene glycol, dipropylene glycol, 1,2-butylene glycol, diethylene glycol monoethyl ether, glycerol, diethyl phthalate, isopropyl myristate, triethyl citrate, and benzyl benzoate.

38. Use of the mixture according to any of the embodiments 25 to 31, as an aroma chemical, or to impart an aroma impression to a composition.

39. The use according to embodiment 38, as a fragrance or to impart a fragrance to a composition.

40. Method of imparting an aroma impression to a composition comprising at least the step of adding the mixture according to any of the embodiment 25 to 31 to a composition to be aromatized, or mixing the mixture according to any of the embodiment 24 to 30 simultaneously or consecutively with the other components of said composition, or mixing the mixture according to any of the embodiment 24 to 30 with a pre-formed mixture of a part of the other components of said composition.

41. The use or method according to any of the embodiment 38 to 40, wherein the aroma impression is selected from a camphoraceous note, citrus note, floral note, herbal note, ethereal note, fresh note, lonone note, spicy note, woody note, carrot like note, juniper note, pepper note, spearmint note, cedarwood note, dandelion note, earthy note, food-like note, or a combination of two or more notes.

42. The use or method according to any of the embodiments 38 to 41, wherein the mixture of compounds of formula (I) and (II) is used in a total amount of the compounds (I) and (II) in the range of ≥0.001 wt. % to ≤70.0 wt. %, based on the total weight of the composition.

### Examples

The present invention is illustrated in detail by non-restrictive working examples which follow. More particularly, the test methods specified hereinafter are part of the general disclosure of the application and are not restricted to the specific working examples.

### Analytical Method:

NMR spectra were recorded on a Bruker DPX500 US 500 MHz spectrometer and referenced to the residual carbon resonance peak of the solvent CDCl₃.

### Gc Analysis:

GC analyses were performed using an Agilent 7890 A equipped with a DB-Wax column (30 m, 0.25 mm, 0.25 μm).

| | |
|---|---|
| Temperature program: | 50° C. |
| | 5 K/min to |
| | 220° C. |
| Constant Flow: | 1.0 ml/min |
| Carrier gas: | Nitrogen |
| Injector temperature: | 220° C. |
| Detector temperature: | 250° C. |

or
GC analyses were performed using an Agilent 7890 A equipped with a CP-Sil 13 CB (50 m, 0.32 mm, 1.2 μm).

| | |
|---|---|
| Temperature program: | 80° C. |
| | 10 K/min to |
| | 250° C. |
| Constant Flow: | 1.15 ml/min |
| Carrier gas: | Nitrogen |
| Injector temperature: | 250° C. |
| Detector temperature: | 280° C. |

### Example 1: Preparation of 3-isopropyl-6-methylcyclohex-2-en-1-ol

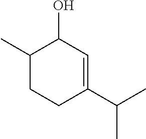

3-Isopropyl-6-methylcyclohex-2-en-1-one (carvenone; 70.0 g, purity 88%, 12% 2-hydroxy-1,4-cineole, 0.403 mol, 1.00 equiv.) was added to tetrahydrofuran (420 mL) and cooled to 0° C. temperature. To this mixture, lithium aluminum hydride (1 M in THF, 182 g, 0.201 mol, 0.50 equiv.) was added within 60 minutes and the mixture was stirred at 0° C. for 2 hours. The mixture was carefully quenched with aqueous hydrochloric acid (10%, 200 mL). Additional water (150 mL) was added to dissolve the forming solids. The mixture was extracted three times with ethyl acetate (200 mL). The combined organic phases were dried over Na₂SO₄ and the solvent was removed under reduced pressure. Two of these batches were combined and purified by distillation (T_{head}=51° C., 0.1 mbar) to yield the title compound (64.1 g) as a colorless liquid in a purity of 89.6 GC-a % as a mixture of isomers in a ratio 64.48:25.07. The mixture contained ca. 10 GC-area-% of 2-hydroxy-1,4-cineole.

¹H NMR (500 MHz, CDCl₃) δ 5.50-5.20 (m, 1H), 3.95-3.57 (m, 1H), 2.58-2.31 (m, 1H), 2.14-1.72 (m, 3H), 1.69-1.13 (m, 6H), 0.97-0.76 (m, 6H).

¹³C NMR (126 MHz, CDCl₃) δ 147.87, 146.26, 122.13, 120.90, 88.58, 85.74, 76.46, 73.40, 67.82, 67.78, 45.13, 37.07, 34.76, 34.48, 33.80, 32.86, 32.43, 32.01, 28.87, 26.21, 25.50, 25.47, 25.18, 21.40, 21.31, 21.07, 18.27, 18.10, 17.99, 16.30, 16.26, 14.09.

### Example 2: Preparation of 5-isopropyl-2-methylcyclohexan-1-ol

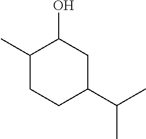

6 g of 3-isopropyl-6-methylcyclohex-en-1-one (carvenone, purity 87.5%, 12% 2-hydroxy-1,4-cineole) were dissolved in 100 ml of ethanol and placed in a 300 ml stainless steel autoclave. 1.8 g of Raney-Nickel (50% moisture) were added to the mixture. Nitrogen was introduced in the autoclave and after heating to 110° C. hydrogen was passed at a pressure of 30 bar on the stirred reaction mixture. The mixture was hydrogenated for 24 h under these conditions.

After depressurization and cooling to room temperature, the catalyst was filtered from the reaction mixture over celite and the solvent was evaporated. Analysis per GC (area %) showed complete conversion of carvenone and as product a mixture of diastereomers in a ratio 47.3:25.8:12.8:14.0 was obtained. The product mixture contained 11.6% of 2-hydroxy-1,4-cineole.

### Example 3: Preparation of 5-isopropyl-2-methylcyclohexan-1-ol

81.8 g of 3-isopropyl-6-methylcyclohex-en-1-one (carvenone, purity 97.3%) were dissolved in 80 ml of ethanol and placed in a 300 ml stainless steel autoclave. 6 g of Raney-Nickel (50% moisture) were added to the mixture. Nitrogen was introduced in the autoclave and after heating to 110° C. hydrogen was passed at a pressure of 30 bar on the stirred reaction mixture. The mixture was hydrogenated for 24 h under these conditions.

After depressurization and cooling down to room temperature the catalyst was filtered from the reaction mixture over celite and the solvent was evaporated. Analysis per GC (area %) showed complete conversion of carvenone and the product with a purity of 98.4% as a mixture of diastereomers in a ratio 37.9:37.43:6.14:17.6 was obtained.

### Example 4: Preparation of 5-isopropyl-2-methylcyclohexan-1-ol

10.4 g of(S)-(+)-carvone were dissolved in 100 ml of ethanol and placed in a 300 ml stainless steel autoclave. 3 g of Raney-Nickel (50% moisture) were added to the mixture. Nitrogen was introduced in the autoclave and after heating to 110° C. hydrogen was passed at a pressure of 30 bar on the stirred reaction mixture. The mixture was hydrogenated for 24 h under these conditions.

After depressurization and cooling down to room temperature the catalyst was filtered from the reaction mixture over celite and the solvent was evaporated. Analysis per GC (area %) showed complete conversion of carvone and the product with a purity of 98.7% as a mixture of diastereomers in a ratio of 37.2:21.8:19.7:21.3 was obtained.

### Example 5: Preparation of 5-isopropyl-2-methylcyclohexan-1-ol

10.2 g of (R)-(−)-carvone were dissolved in 100 ml of ethanol and placed in a 300 ml stainless steel autoclave. 3 g of Raney-Nickel (50% moisture) were added. Nitrogen was introduced in the autoclave and after heating to 110° C. hydrogen was passed at a pressure of 30 bar on the stirred reaction mixture. The mixture was hydrogenated for 24 h under these conditions.

After depressurization the catalyst was filtered from the reaction mixture over celite and the solvent was evaporated. Analysis per GC (area %) showed complete conversion of carvone and the product with a purity of 99.1% as a mixture of diastereomers in a ratio of 36.5:21.7:19.6:22.1 was obtained.

### Example 6: Preparation of (5-isopropyl-2-methyl-cyclohexyl)acetate

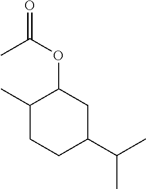

5-Isopropyl-2-methyl-cyclohexanol (20.0 g, 0.128 mol, 1.00 equiv.) and N,N-dimethylpyridin-4-amine (1.25 g, 0.01 mol, 0.08 equiv.) were added to tetrahydrofuran (60 mL) and heated to 60° C. To this mixture, acetic anhydride (16.3 g, 0.160 mol, 1.25 equiv.) was added within 30 minutes and the mixture was stirred at 60° C. for 5 hours. After cooling to room temperature, the mixture was diluted with ethyl acetate (40 mL) and quenched with aqueous saturated NaHCO₃ solution (200 mL). The phases were separated, the organic phase was washed with aqueous saturated NaHCO₃ solution (20 mL) and NaCl solution (15 mL), dried over Na₂SO₄ and the solvent was removed under reduced pressure. The crude product was purified by distillation (T_{head}=62° C., 1.2 mbar) to yield the title compound (12.0 g) as a colorless liquid in a purity of 99.6 GC-a % as a mixture of isomers in a ratio 18.09:40.21:6.31:34.96.

¹H NMR (500 MHz, CDCl₃) δ 5.00-4.27 (m, 1H), 2.03-1.90 (m, 2H), 1.88-0.91 (m, 10H), 0.89-0.76 (m, 9H).

¹³C NMR (126 MHz, CDCl₃) δ 170.87, 170.81, 170.61, 170.47, 78.75, 75.61, 75.04, 73.89, 42.89, 42.67, 39.36, 37.43, 37.28, 35.31, 35.30, 34.13, 33.85, 33.18, 32.57, 32.53, 32.37, 31.09, 30.69, 30.38, 30.27, 29.17, 28.97, 28.89, 28.78, 27.60, 24.59, 22.58, 21.42, 21.38, 21.29, 21.22, 20.33, 20.23, 19.85, 19.81, 19.74, 19.73, 19.49, 18.30, 18.10, 17.13, 11.60.

### Example 7: Preparation of (5-isopropyl-2-methyl-cyclohexyl)acetate

5 g of(S)-(+)-2-methyl-5-iso-propyl-cyclohexanol (carvomenthol, purity 97%, coming from Example 4) were mixed with 6.9 g of acetic acid anhydride, 400 mg of nafion ion exchanger and 0.9 g of acetic acid. The mixture was stirred for 92 h at room temperature. The mixture was then filtrated and 100 ml methyl-tert-butyl ether was added. The organic solution was washed with water and the layers were separated. The aqueous layer was extracted a second time with methyl-tert-butyl ether and the combined organic layers were then washed with a saturated NaHCO₃ solution. After washing with brine, the organic layer was dried over MgSO₄ and the solvent was evaporated, yielding 4.3 g of the desired product with 97% purity as a mixture of diastereoisomers in the ratio 20.8:21.8:19.7:37.7.

### Example 8: Preparation of (5-isopropyl-2-methyl-cyclohexyl)acetate

5 g of (R)-(−)-2-methyl-5-iso-propyl-cyclohexanol (carvomenthol, purity 99%, from Example 5) were mixed with 6.6 g of acetic acid anhydride, 400 mg of nafion ionexchanger and 0.4 g of acetic acid. The mixture was stirred for 69 h at room temperature. The mixture was then filtrated and 100 ml methyl-tert-butyl ether was added. The organic solution was washed with water and the layers were separated. The organic layer was extracted a second time with methyl-tert-butyl ether and the combined organic layers were then washed with a saturated NaHCO₃ solution. After washing with brine the organic layer was dried over MgSO₄, the solvent was evaporated and the crude material was distilled under vacuum, yielding 3.3 g of the desired product with a purity of 98.7% as a mixture of diastereoisomers in the ratio: 13.2:2.3:22.0:43.5.

### Example 9: Preparation of 3-isopropyl-6-methylcyclohex-2-en-1-yl acetate

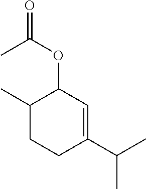

3-Isopropyl-6-methylcyclohex-2-en-1-ol (13.5 g, 0.88 mol, 1.00 equiv.) and N,N-dimethylpyridin-4-amine (0.855 g, 0.007 mol, 0.08 equiv.) was mixed. To this mixture, acetic anhydride (11.2 g, 0.109 mol, 1.25 equiv.) was added within 20 minutes and the mixture was stirred at 60° C. for 2 hours. After cooling to room temperature, the mixture was diluted with ethyl acetate (40 mL) and quenched with aqueous saturated NaHCO₃ solution (15 mL). The phases were separated, the organic phase was washed twice with aqueous saturated NaHCO₃ solution (15 mL) and NaCl solution (15 mL), dried over Na₂SO₄ and the solvent was removed under reduced pressure. The crude product was purified by column chromatography over SiO₂ (cyclohexane/ethyl acetate 95:5 to 90:10) to yield the title compound (4.40 g) as a colorless liquid in a purity of 97.2 GC-a % as a mixture of isomers.

¹H NMR (500 MHz, CDCl₃) δ 5.51-5.20 (m, 1H), 5.18-4.86 (m, 1H), 2.13 (dt, J=13.8, 6.9 Hz, 1H), 2.04-1.86 (m, 4H), 1.79-1.60 (m, 2H), 1.53-1.26 (m, 2H), 1.05-0.73 (m, 9H).

¹³C NMR (126 MHz, CDCl₃) δ 171.16, 170.99, 149.93, 148.58, 117.79, 117.12, 75.98, 70.84, 34.88, 34.71, 33.50, 32.16, 28.43, 26.90, 26.12, 26.03, 25.02, 21.40, 21.39, 21.24, 21.22, 21.07, 17.87, 16.24.

### Example 10: Preparation of 3-ethoxy-1-isopropyl-4-methylcyclohex-1-ene

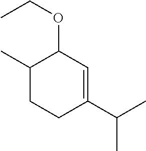

Sodium hydride (60% dispersion in mineral oil, 3.90 g, 0.0972 mol, 1.50 equiv.) was added to tetrahydrofuran (25 mL). 3-Isopropyl-6-methylcyclohex-2-en-1-ol (10.0 g, 0.0648 mol, 1.00 equiv.) in tetrahydrofuran (15 mL) was added to the mixture within 15 minutes. The resulting mixture was stirred for additional 30 minutes at room temperature. Iodoethane (24.0 g, 0.154 mol, 1.20 equiv.) was added within 8 minutes. The mixture was heated to 40° C. and stirred for 5 hours. Additional iodoethane (0.50 equiv.) was added and the mixture was stirred at 40° C. for additional 18 hours. The reaction mixture was cooled to room temperature and quenched by addition of water (25 mL). The mixture was extracted twice with 2-methoxy-2-methylpropane (30 mL). The combined organic layers were washed three times with ammonia solution (20 mL) and once with saturated NaCl solution (20 mL) and dried over Na₂SO₄ and the solvent was removed under reduced pressure. The crude product was purified by column chromatography over SiO₂ (cyclohexane/ethyl acetate 975:25 to 95:5) to yield the title compound (1.40 g) as a colorless liquid in a purity of 98.2 GC-a % as a mixture of isomers.

¹H NMR (500 MHz, CDCl₃) δ 7.29-7.22 (m, 1H), 5.60-5.15 (m, 3H), 4.09-2.95 (m, 12H), 2.85-2.69 (m, 6H).

¹³C NMR (126 MHz, CDCl₃) δ 146.83, 146.44, 119.49, 119.21, 80.99, 76.36, 64.37, 63.77, 34.94, 34.89, 34.01, 31.54, 28.98, 27.00, 26.62, 25.32, 24.45, 21.47, 21.40, 21.37, 21.23, 18.43, 15.87, 15.81, 14.14.

### Example 11: Preparation of (5-isopropyl-2-methyl-cyclohexyl) propanoate

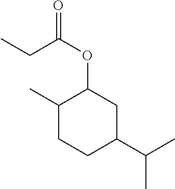

5-Isopropyl-2-methyl-cyclohexanol (20.0 g, 0.128 mol, 1.00 equiv.) and N,N-dimethylpyridin-4-amine (1.25 g, 0.01 mol, 0.08 equiv.) were added to tetrahydrofuran (60 mL) and heated to 60° C. To this mixture, propionic anhydride (20.8 g, 0.160 mol, 1.25 equiv.) was added within 30 minutes and the mixture was stirred at 60° C. for 5 hours. After cooling to room temperature, the mixture was diluted with ethyl acetate (40 mL) and quenched with aqueous saturated NaHCO₃ solution (200 mL). The phases were separated, the organic phase was washed with aqueous saturated NaHCO₃ solution (20 mL) and NaCl solution (15 mL), dried over Na₂SO₄ and the solvent was removed under reduced pressure. The crude product was purified by distillation (T_{head}=84° C., 2.5 mbar) to yield the title compound (11.8 g) as a colorless liquid in a purity of 99.7 GC-a % as a mixture of isomers in a ratio 18.47:41.66:5.99:33.55.

¹H NMR (500 MHz, CDCl₃) δ 5.06-4.37 (m, 1H), 2.39-2.27 (m, 2H), 2.21-0.96 (m, 12H), 0.96-0.79 (m, 9H).

¹³C NMR (126 MHz, CDCl₃) δ 174.33, 174.31, 174.12, 173.97, 78.59, 75.43, 74.88, 73.69, 42.93, 42.73, 39.46, 37.52, 37.40, 35.42, 35.36, 34.16, 33.95, 33.25, 32.61, 32.58, 32.42, 31.21, 30.75, 30.43, 30.32, 29.30, 29.04, 28.85, 28.16, 28.14, 28.10, 28.04, 27.70, 24.71, 22.66, 20.38, 20.34, 19.92, 19.84, 19.80, 19.62, 18.36, 18.18, 17.20, 11.69, 9.55, 9.40, 9.35.

### Example 12: Preparation of 4-isopropyl-2-methoxy-1-methyl-cyclohexane

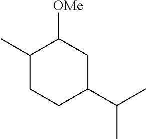

Sodium hydride (60% dispersion in mineral oil, 6.14 g, 0.154 mol, 1.20 equiv.) was added to tetrahydrofuran (100 mL). 5-Isopropyl-2-methyl-cyclohexanol (20.0 g, 0.128 mol, 1.00 equiv.) in tetrahydrofuran (20 mL) was added to the mixture within 15 minutes. The resulting mixture was stirred for additional 30 minutes at room temperature. Iodomethane (21.8 g, 0.154 mol, 1.20 equiv.) was added within 10 minutes. The mixture was heated to 40° C. and stirred for 3 hours. The reaction mixture was cooled to room temperature and quenched by the addition of water (50 mL). The mixture was extracted three times with 2-methoxy-2-methylpropane (60 mL). The combined organic layers were washed three times with ammonia solution (40 mL) and once with saturated NaCl solution (40 mL) and dried over Na₂SO₄ and the solvent was removed under reduced pressure. The crude product was purified by distillation (T_{head}=45° C., 1.7 mbar) to yield the title compound (11.0 g) as a colorless liquid in a purity of 99.7 GC-a % as a mixture of isomers in a ratio 18.79:6.68:40.62:33.72.

¹H NMR (500 MHz, CDCl₃) δ 3.37-3.29 (m, 3H), 3.28-3.06 (m, 1H), 2.29-1.03 (m, 7H), 0.99-0.92 (m, 2H), 0.90-0.83 (m, 9H).

¹³C NMR (126 MHz, CDCl₃) δ 85.92, 81.98, 81.58, 80.56, 56.76, 56.52, 56.04, 55.52, 43.28, 43.03, 38.64, 38.58, 36.79, 36.73, 34.14, 33.76, 32.89, 32.84, 32.79, 32.52, 31.97, 31.18, 30.65, 30.00, 29.56, 29.33, 29.18, 29.13, 29.04, 27.36, 24.36, 23.07, 20.19, 20.12, 19.98, 19.93, 19.87, 19.82, 19.77, 18.75, 18.49, 17.34, 10.80.

### Example 13: Preparation of 2-ethoxy-4-isopropyl-1-methyl-cyclohexane

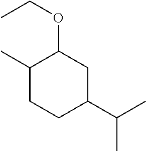

Sodium hydride (60% dispersion in mineral oil, 6.14 g, 0.154 mol, 1.20 equiv.) was added to tetrahydrofuran (100 mL). 5-Isopropyl-2-methyl-cyclohexanol (20.0 g, 0.128 mol, 1.00 equiv.) in tetrahydrofuran (20 mL) was added to the mixture within 15 minutes. The resulting mixture was stirred for additional 30 minutes at room temperature. Iodoethane (24.0 g, 0.154 mol, 1.20 equiv.) was added within 10 minutes. The mixture was heated to 40° C. and stirred for 5 hours. Additional iodoethane (0.50 equiv.) was added and the mixture was stirred at 40° C. for additional 18 hours. The reaction mixture was cooled to room temperature and quenched by the addition of water (50 mL). The mixture was extracted three times with 2-methoxy-2-methylpropane (60 mL). The combined organic layers were washed three times with ammonia solution (40 mL) and once with saturated NaCl solution (40 mL) and dried over Na₂SO₄ and the solvent was removed under reduced pressure. The crude product was purified by distillation (T_{head}=60° C., 2.5 mbar) and column chromatography over SiO₂ (cyclohexane/ethyl acetate 95:5 to 90:10) to yield the title compound (3.00 g) as a colorless liquid in a purity of 99.6 GC-a % as a mixture of isomers in a ratio 33.35:9.64:56.60.

¹H NMR (500 MHz, CDCl₃) δ 3.88-3.08 (m, 3H), 2.25-1.02 (m, 12H), 0.98-0.80 (m, 9H).

¹³C NMR (126 MHz, CDCl₃) δ 84.32, 80.20, 79.63, 78.45, 72.42, 71.35, 64.26, 63.59, 62.82, 43.38, 43.13, 39.05, 38.67, 37.12, 36.88, 36.72, 36.59, 36.50, 35.12, 34.37, 33.77, 33.10, 32.84, 32.78, 32.60, 32.56, 30.66, 30.45, 30.37, 30.35, 30.16, 29.35, 29.26, 29.19, 29.01, 28.31, 27.75, 27.47, 24.86, 23.14, 20.56, 20.49, 20.42, 20.31, 20.03, 20.01, 19.95, 19.92, 19.91, 19.86, 19.85, 19.77, 19.76, 19.69, 18.80, 18.46, 18.46, 17.66, 15.89, 15.85, 15.82, 15.79, 10.98.

### Example 14: Preparation of (4-isopropyl-1-methyl-7-oxabicyclo[2.2.1]heptan-2-yl)acetate

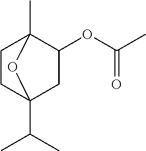

4-Isopropyl-1-methyl-7-oxabicyclo[2.2.1]heptan-2-ol (50.0 g, 0.294 mol, 1.00 equiv.) and N,N-dimethylpyridin-4-amine (2.87 g, 0.023 mol, 0.08 equiv.) were added to tetrahydrofuran (200 mL) and heated to 60° C. To this mixture, acetic anhydride (37.5 g, 0.367 mol, 1.25 equiv.) was added and the mixture was stirred at 60° C. for 5 hours. After cooling to room temperature, the mixture was diluted with ethyl acetate (200 mL) and quenched with aqueous saturated NaHCO₃ solution (100 mL). The phases were separated, the organic phase was washed with aqueous saturated NaHCO₃ solution (3×100 mL) and NaCl solution (100 mL), dried over Na₂SO₄ and the solvent was removed under reduced pressure. The crude product was purified by distillation (T_{head}=95° C., 1.0 mbar) to yield the title compound (50.4 g) as a colorless liquid in a purity of 99.9 GC-a % as a mixture of isomers.

¹H NMR (500 MHz, CDCl₃) δ 4.88 (dd, J=7.3, 2.6 Hz, 1H), 2.18 (dd, J=13.2, 7.3 Hz, 1H), 2.13-2.08 (m, 1H), 2.07 (s, 3H), 1.65-1.43 (m, 5H), 1.39 (s, 3H), 1.02-0.92 (m, 6H).

¹³C NMR (126 MHz, CDCl₃) δ 170.8, 88.8, 84.4, 78.4, 77.4, 77.2, 76.9, 43.3, 33.4, 32.5, 31.2, 21.2, 18.2, 17.9, 16.

### Example 15: Preparation of 2-ethoxy-4-isopropyl-1-methyl-7-oxabicyclo[2.2.1]heptane

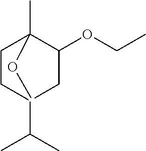

Sodium hydride (60% dispersion in mineral oil, 7.05 g, 0.176 mol, 1.50 equiv.) was added to tetrahydrofuran (50 mL). 4-Isopropyl-1-methyl-7-oxabicyclo[2.2.1]heptan-2-ol (20.0 g, 0.117 mol, 1.00 equiv.) in tetrahydrofuran (30 mL) was added to the mixture within 15 minutes. The resulting mixture was stirred for additional 30 minutes at room temperature. Iodoethane (27.5 g, 0.176 mol, 1.50 equiv.) was added within 10 minutes. The mixture was heated to 40° C. and stirred for 23 hours. The reaction mixture was cooled to room temperature and quenched by the addition of water (50 mL). The mixture was extracted twice with 2-methoxy-2-methylpropane (60 mL). The combined organic layers were washed three times with ammonia solution (40 mL) and once with saturated NaCl solution (40 mL) and dried over Na₂SO₄ and the solvent was removed under reduced pressure. The crude product was purified by distillation (T_{head}=86-95° C., 2.0 mbar) and column chromatography over SiO₂ (cyclohexane/ethyl acetate 95:5 to 90:10) to yield the title compound (7.80 g) as a colorless liquid in a purity of 99.9 GC-a % as a mixture of isomers.

¹H NMR (500 MHz, CDCl₃) δ 3.53-3.43 (m, 2H), 3.35 (dq, J=9.3, 7.0 Hz, 1H), 2.09 (h, J=6.9 Hz, 1H), 1.96 (dd, J=12.5, 6.8 Hz, 1H), 1.55 (ddd, J=8.7, 6.6, 3.0 Hz, 2H), 1.50-1.40 (m, 6H), 1.18 (t, J=7.0 Hz, 3H), 0.97 (dd, J=6.9, 2.5 Hz, 6H).

¹³C NMR (126 MHz, CDCl₃) δ 88.5, 85.4, 83.6, 64.1, 42.7, 33.9, 32.7, 31.7, 26.9, 18.3, 18.0, 16.5, 15.4.

### Example 16: Preparation of 5-isopropyl-2-methylcyclohexyl 2-methylpropanoate

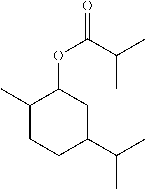 [SMILES: CC(C)C(=O)OC1CC(C(C)C)CCC1C]

5-Isopropyl-2-methyl-cyclohexanol (5.00 g, 0.032 mol, 1.00 equiv.) and N,N-dimethylpyridin-4-amine (0.313 g, 2.56 mmol, 0.08 equiv.) were added to tetrahydrofuran (20 mL) and heated to 60° C. To this mixture, isobutyric anhydride (6.33 g, 0.040 mol, 1.25 equiv.) was added within 30 minutes and the mixture was stirred at 60° C. for 3 hours.

After cooling to room temperature, the mixture was diluted with ethyl acetate (30 mL) and quenched with aqueous saturated NaHCO₃ solution (20 mL). The phases were separated, the organic phase was washed with aqueous saturated NaHCO₃ solution (2×20 mL) and NaCl solution (10 mL), dried over Na₂SO₄ and the solvent was removed under reduced pressure. The crude product was purified by column chromatography over SiO₂ (cyclohexane/ethyl acetate 95:5 to 90:10) to yield the title compound (4.50 g) as a colorless liquid in a purity of 99.4 GC-a % as a mixture of isomers in a ratio 18.08:37.3:6.36:37.64.

¹H NMR (500 MHz, CDCl₃) δ 5.03-4.37 (m, 1H), 2.64-2.43 (m, 1H), 2.20-1.22 (m, 9H), 1.19-1.13 (m, 6H), 0.96-0.80 (m, 9H).

¹³C NMR (126 MHz, CDCl₃) δ 177.03, 176.92, 176.79, 176.69, 78.43, 75.27, 74.72, 73.53, 42.95, 42.77, 39.52, 37.58, 37.56, 35.49, 35.34, 34.69, 34.47, 34.46, 34.03, 33.86, 33.32, 32.64, 32.62, 32.45, 31.25, 30.57, 30.46, 29.45, 29.29, 29.03, 28.88, 27.77, 27.05, 24.79, 22.71, 20.43, 20.36, 19.98, 19.89, 19.85, 19.82, 19.77, 19.33, 19.31, 19.29, 19.23, 19.21, 19.17, 19.11, 18.37, 18.23, 17.20, 11.73.

### Example 17: Preparation of 4-isopropyl-1-methyl-7-oxabicyclo[2.2.1]heptan-2-yl 2-methylpropanoate

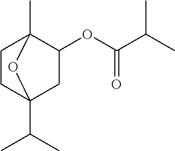 [SMILES: CC(C)C(=O)OC1CC2(C(C)C)CCC1(C)O2]

4-Isopropyl-1-methyl-7-oxabicyclo[2.2.1]heptan-2-ol (5.00 g, 0.0294 mol, 1.00 equiv.) and N,N-dimethylpyridin-4-amine (0.287 g, 2.35 mmol, 0.08 equiv.) were added to tetrahydrofuran (20 mL) and heated to 60° C. To this mixture, isobutyric anhydride (5.81 g, 0.0367 mol, 1.25 equiv.) was added within 30 minutes and the mixture was stirred at 60° C. for 3 hours. After cooling to room temperature, the mixture was diluted with ethyl acetate (30 mL) and quenched with aqueous saturated NaHCO₃ solution (20 mL). The phases were separated, the organic phase was washed with aqueous saturated NaHCO₃ solution (2×20 mL) and NaCl solution (10 mL), dried over Na₂SO₄ and the solvent was removed under reduced pressure. The crude product was purified by column chromatography over SiO₂ (cyclohexane/ethyl acetate 90:10) to yield the title compound (4.50 g) as a colorless liquid in a purity of >99.9 GC-a % as a mixture of isomers.

¹H NMR (500 MHz, CDCl₃) δ 4.84 (dd, J=7.2, 2.6 Hz, 1H), 2.56 (p, J=7.0 Hz, 1H), 2.11 (ddd, J=33.3, 13.4, 7.0 Hz, 2H), 1.66-1.41 (m, 5H), 1.37 (s, 3H), 1.16 (dd, J=8.5, 7.0 Hz, 6H), 0.96 (dd, J=6.9, 5.1 Hz, 6H).

¹³C NMR (126 MHz, CDCl₃) δ 177.05, 88.87, 84.61, 78.11, 43.28, 34.17, 33.71, 32.57, 31.60, 19.21, 19.03, 18.26, 18.11, 16.41.

### Example 18: Mixture of (5-isopropyl-2-methyl-cyclohexyl) propanoate and 4-isopropyl-1-methyl-7-oxabicyclo[2.2.1]heptan-2-yl) propanoate

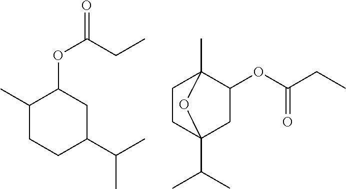

A ~85:12 GC-a % mixture of 5-isopropyl-2-methyl-cyclohexanol and 4-isopropyl-1-methyl-7-oxabicyclo[2.2.1]heptan-2-ol (30.0 g) and N,N-dimethylpyridin-4-amine (1.88 g, 0.015 mol) were added to tetrahydrofuran (90 mL) and heated to 60° C. To this mixture, propionic anhydride (31.2 g, 0.240 mol) was added within 30 minutes and the mixture was stirred at 60° C. for 3 hours. After cooling to room temperature, the mixture was diluted with ethyl acetate (60 mL) and quenched with aqueous saturated NaHCO₃ solution (300 mL). The phases were separated, the organic phase was washed with aqueous saturated NaHCO₃ solution (30 mL) and NaCl solution (20 mL), dried over Na₂SO₄ and the solvent was removed under reduced pressure. The crude product was purified by distillation (T_{head}=72° C., 1.5 mbar) to yield the title compound (25.4 g) as a colorless liquid in a total purity of >99 GC-a % as a mixture of isomers in a ratio of (5-isopropyl-2-methyl-cyclohexyl) propanoate to 4-isopropyl-1-methyl-7-oxabicyclo[2.2.1]heptan-2-yl) propanoate from 93:6 to 88:11.

### Example 19: Mixture of 2-methoxy-4-isopropyl-1-methyl-cyclohexane and 2-methoxy-4-isopropyl-1-methyl-7-oxabicyclo[2.2.1]heptane

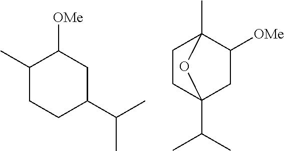

Sodium hydride (60% dispersion in mineral oil, 9.21 g, 0.154 mol) was added to tetrahydrofuran (110 mL). A ~85:12 GC-a % mixture of 5-isopropyl-2-methyl-cyclohexanol and 4-isopropyl-1-methyl-7-oxabicyclo[2.2.1]heptan-2-ol (30.0 g) in tetrahydrofuran (40 mL) was added to the mixture within 15 minutes. The resulting mixture was stirred for additional 30 minutes at room temperature. Iodomethane (32.7 g, 0.230 mol) was added within 10 minutes. The mixture was heated to 40° C. and stirred for 2 hours. The reaction mixture was cooled to room temperature and quenched by the addition of water (75 mL). The mixture was extracted three times with 2-methoxy-2-methylpropane (80 mL). The combined organic layers were washed three times with ammonia solution (60 mL) and once with saturated NaCl solution (40 mL) and dried over Na₂SO₄ and the solvent was removed under reduced pressure. The crude product was purified by distillation (T_{head}=46° C., 1.8 mbar) to yield the title compound (22.6 g) as a colorless liquid in a purity of >99 GC-a % as a mixture of isomers in a ratio of 2-methoxy-4-isopropyl-1-methyl-cyclohexane to 2-methoxy-4-isopropyl-1-methyl-7-oxabicyclo[2.2.1]heptane from 97:3 to 90:10.

Other mixtures were prepared by mixing the respective compounds (I) and (II) in the weight ratio indicated in Table I.

### 3. Olfactory Evaluation

In order to test the quality and intensity of the compounds of the present invention, scent strip tests were performed.

For this purpose, strips of absorbent paper were dipped into a solution containing 1 to 10 wt. % of the compound to be tested in triethyl citrate. After evaporation of the solvent (about 30 s) the scent impression was olfactively evaluated by a trained perfumer.

TABLE I

| Mixture/compound | Ratio | Olfactory Impression |
|---|---|---|
| 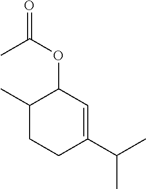 | — | Floral-Ionone Fruity Powdery Animalic-Ambrinol Anisic Fatty-Creamy Floral-Heliotrope Fruity |
| 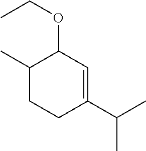 | — | Anisic-Licorice Citrus-Agrumic Floral Gourmand-Sweet Herbal Technical-Ethereal Floral-Violet Floral-Heliotrope Fruity-Apple Fruity-Pear |
| 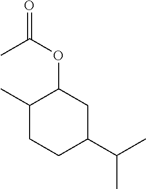 | — | Floral-Violet Powdery Fatty-Soapy Minty-Spearmint |
| 5-isopropyl-2-methyl- | | |
| cyclohexyl) acetate | | |
| 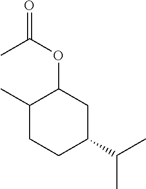 | — | Floral-Violet- Fruity-Raspberry- Gourmand-Dried-Fruit |
| (5R)-5-isopropyl-2- | | |
| methylcyclohexyl acetate | | |
| 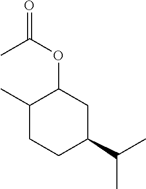 | — | Musty-Dusty Musty-Dry Woody-Cedarwood |
| (5S)-5-isopropyl-2- | | |
| methylcyclohexyl acetate | | |
| 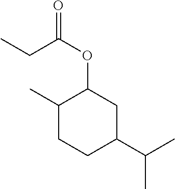 | — | Floral Powdery Warm Woody Fruity Tobacco |
| (5-isopropyl-2-methyl- | | |
| cyclohexyl) propanoate | | |
| 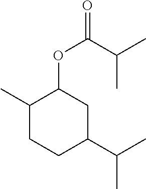 [SMILES: CC(C)C(=O)OC1CC(C(C)C)CCC1C] | — | Fresh Minty Agrumic |
| 5-isopropyl-2-methylcyclo- | | |
| hexyl 2-methylpropanoate | | |
| 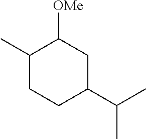 | — | Fresh Minty |
| 4-isopropyl-2-methoxy-1- | | |
| methyl-cyclohexane | | |
| 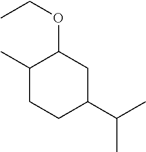 | — | Food Like-Carrot Natural Rooty Fatty-Creamy Gourmand-Raisin Green |
| 2-ethoxy-4-isopropyl-1- | | |
| methyl-cyclohexane | | |
| 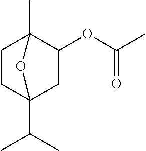 | — | Ambrinol Animalic Civet soft |
| 4-isopropyl-1-methyl-7- | | |
| oxabicyclo[2.2.1]heptan-2-yl) | | |
| acetate | | |
| 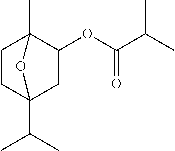 [SMILES: CC(C)C(=O)OC1CC2(C(C)C)CCC1(C)O2] | — | |
| 4-isopropyl-1-methyl-7-oxa- | | |
| bicyclo[2.2.1]heptan-2-yl) 2- | | |
| methylpropanoate | | |
| 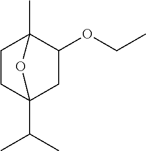 | — | Herbal Incense |
| 2-ethoxy-4-isopropyl-1- | | |
| methyl-7- | | |
| oxabicyclo[2.2.1]heptane | | |
| Mixture 1 | 3-ethoxy-1-isopropyl-4- | Camphoraceous |
| 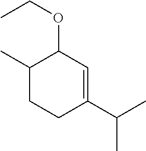 | methylcyclohex-1-ene: 4-isopropyl-1-methyl-7- oxabicyclo[2.2.1]heptan- 2-yl) acetate 85:15 | Citrus-Bergamot Floral-Damascone Herbal Technical-Ethereal |
| 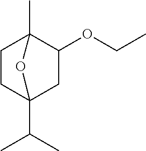 | | |
| Mixture of 3-ethoxy-1- | | |
| isopropyl-4-methylcyclohex- | | |
| 1-ene and 4-isopropyl-1- | | |
| methyl-7- | | |
| oxabicyclo[2.2.1]heptan-2-yl) | | |
| acetate | | |
| Mixture 2 | 5-isopropyl-2-methyl- | Floral |
| 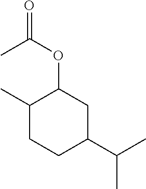 | cyclohexyl) acetate: 4- isopropyl-1-methyl-7- oxabicyclo[2.2.1]heptan- 2-yl) acetate 88:12 | Fresh Ionone like Spicy Woody |
| 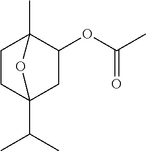 | | |
| Mixture of 5-isopropyl-2- | | |
| methyl-cyclohexyl) acetate | | |
| and 4-isopropyl-1-methyl-7- | | |
| oxabicyclo[2.2.1]heptan-2-yl) | | |
| acetate | | |
| Mixture 3 | 2-ethoxy-4-isopropyl-1- | Spicy |
| 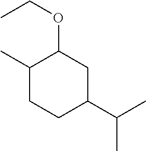 | methyl-cyclohexane: 2- ethoxy-4-isopropyl-1- methyl-7- oxabicyclo[2.2.1]heptane 88:12 | Food like Carrot like Fresh Ethereal |
| 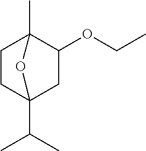 | | |
| Mixture of 2-ethoxy-4- | | |
| isopropyl-1-methyl-cyclo- | | |
| hexane and 2-ethoxy-4- | | |
| isopropyl-1-methyl-7- | | |
| oxabicyclo[2.2.1]heptane | | |
| Mixture 4 | 5-isopropyl-2-methyl- | Slightly spicy |
| 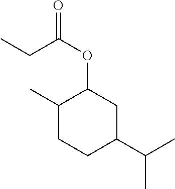 | cyclohexyl) propanoate: 4-isopropyl-1-methyl-7- oxabicyclo[2.2.1]heptan- 2-yl) propanoate 93:7 | Juniper Pepper Woody Warm Spearmint |
| 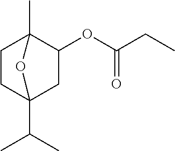 [SMILES: CCC(=O)OC1CC2(C(C)C)CCC1(C)O2] | | |
| Mixture of 5-isopropyl-2- | | |
| methyl-cyclohexyl) | | |
| propanoate and 4-isopropyl- | | |
| 1-methyl-7- | | |
| oxabicyclo[2.2.1]heptan-2-yl) | | |
| propanoate | | |
| Mixture 5 | 5-isopropyl-2-methyl- | similar profile to the 93:7 |
| 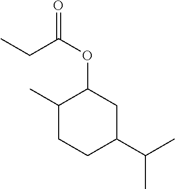 | cyclohexyl) propanoate: 4-isopropyl-1-methyl-7- oxabicyclo[2.2.1]heptan- 2-yl) propanoate 88:11 | mixture 4; a bit more cedarwoody and spicy |
| 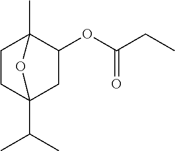 [SMILES: CCC(=O)OC1CC2(C(C)C)CCC1(C)O2] | | |
| Mixture of 5-isopropyl-2- | | |
| methyl-cyclohexyl) | | |
| propanoate and 4-isopropyl- | | |
| 1-methyl-7- | | |
| oxabicyclo[2.2.1]heptan-2-yl) | | |
| propanoate | | |
| Mixture 6 | 5-isopropyl-2-methyl- | Fresh |
| 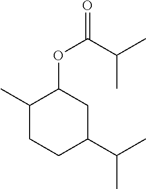 [SMILES: CC(C)C(=O)OC1CC(C(C)C)CCC1C] | cyclohexyl) isobutyrate: 4-isopropyl-1-methyl-7- oxabicyclo[2.2.1]heptan- 2-yl) isobutyrate 89:11 | Minty Dandelion |
| 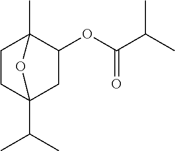 [SMILES: CC(C)C(=O)OC1CC2(C(C)C)CCC1(C)O2] | | |
| Mixture of 5-isopropyl-2- | | |
| methyl-cyclohexyl) | | |
| isobutyrate and 4-isopropyl- | | |
| 1-methyl-7- | | |
| oxabicyclo[2.2.1]heptan-2-yl) | | |
| isobutyrate | | |
| Mixture 7 | 2-methoxy-4-isopropyl- | Minty |
| 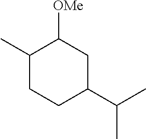 | 1-methyl-cyclohexane: 2-methoxy-4-isopropyl- 1-methyl-7- oxabicyclo[2.2.1]heptane 94:5 | |
| 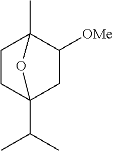 | | |
| Mixture of 2-methoxy-4- | | |
| isopropyl-1-methyl- | | |
| cyclohexane and 2-methoxy- | | |
| 4-isopropyl-1-methyl-7- | | |
| oxabicyclo[2.2.1]heptane | | |
| Mixture 8 | 2-methoxy-4-isopropyl- | Minty |
| 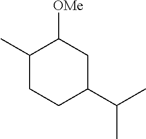 | 1-methyl-cyclohexane: 2-methoxy-4-isopropyl- 1-methyl-7- oxabicyclo[2.2.1]heptane 90:10 | Musty-Earthy |
| 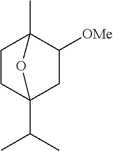 | | |
| Mixture of 2-methoxy-4- | | |
| isopropyl-1-methyl- | | |
| cyclohexane and 2-methoxy- | | |
| 4-isopropyl-1-methyl-7- | | |
| oxabicyclo[2.2.1]heptane | | |

### Advantageous Compositions

The mixtures/compounds indicated in table I were formulated as compositions according to tables 2 and 3. Mixtures/compounds from table I are labelled as “Compound P” in table 2 and 3.

TABLE II
Compositions 1A and 1B

| | 1A | 1B |
|---|---|---|
| Lactone C10 gamma (5-hexyloxolan-2-one) | 2 | 2 |
| Bourgeonal (3-(4-tert-butylphenyl)propanal) | 2 | 2 |
| Citronellol | 3 | 3 |
| Aldehyde C-14 (5-heptyloxolan-2-one) | 3 | 3 |
| Allyl heptylate | 4 | 4 |
| Amber core (1-(2-tert-butylcyclohexyl) oxybutan-2-ol) | 4 | 4 |
| Ethyl-2-methyl butyrate | 4 | 4 |
| Geranyl acetate | 5 | 5 |
| Helional (3-(1,3-benzodioxol-5-yl)-2-methylpropanal) | 10 | 10 |
| Manzanate (ethyl 2-methylpentanoate) | 10 | 10 |
| Amberwood (ethoxymethoxycyclododecane) | 10 | 10 |
| Hexyl acetate | 11 | 11 |
| Benzyl salicylate | 12 | 12 |
| Magnolan (2,4-dimethyl-4,4a,5,9b-tetrahydroindeno[1,2- | 15 | 15 |
| d][1,3]dioxine) | | |
| Verdox (2-tert-butylcyclohexyl) acetate) | 25 | 25 |
| Bergamot oil bergaptene free | 25 | 25 |
| Linalol | 30 | 30 |
| Dipropylene glycol | 45 | 45 |
| Iso E Super (Tetramethyl acetyloctahydronaphthalenes) | 110 | 110 |
| Pyranol (4-methyl-2-(2-methylpropyl)oxan-4-ol) | 170 | 170 |
| Hedione (methyl 3-oxo-2-pentylcyclopentaneacetate) | 200 | 200 |
| Galaxolide 50% IPM (1,3,4,6,7,8-hexahydro-4,6,6,7,8,8- | 300 | 300 |
| hexamethylcyclopenta(g)-2-benzopyran 50% in isopropyl | | |
| myristate) | | |
| compound P | 20 | 50 |
| Total ( part by weight) | 1020 | 1050 |

TABLE III
Compositions 2A and 2B

| | 2A | 2B |
|---|---|---|
| Raspberry ketone (4-(4-hydroxyphenyl)butan-2-one) | 4 | 4 |
| Vanitrope (2-ethoxy-5-prop-1-enylphenol) | 6 | 6 |
| Cyclamen aldehyde (at least 90% 2-methyl-3-(p- | 10 | 10 |
| isopropylphenyl) propionaldehyde; secondary component: | | |
| 5% 3-(p-cumenyl)-2-methylpropionic acid) | | |
| Bicyclononalactone (3,4,4a,5,6,7,8,8a-octahydrochromen- | 10 | 10 |
| 2-one) | | |
| Aldehyde C-14 (5-heptyloxolan-2-one) | 14 | 14 |
| Ethylvanillin (3-ethoxy-4-hydroxybenzaldehyde) | 16 | 16 |
| Heliotropine (1,3-benzodioxole-5-carbaldehyde) | 20 | 20 |
| Iso E Super (tetramethyl acetyloctahydronaphthalenes) | 20 | 20 |
| Sandela (3-[5,5,6-trimethylbicyclo[2.2.1]hept-2-yl] | 30 | 30 |
| cyclohexan-1-ol) | | |
| Vanillin isobutyrate ((4-formyl-2-methoxyphenyl) 2- | 40 | 40 |
| methylpropanoate) | | |
| Aldehyde C-18 (5-pentyloxolan-2-one) | 50 | 50 |
| Benzyl salicylate | 60 | 60 |
| Hexyl cinnamic aldehyde (2-(phenylmethylidene)octanal) | 70 | 70 |
| Hedione (methyl 3-oxo-2-pentylcyclopentaneacetate) | 130 | 130 |
| Pyranol (4-methyl-2-(2-methylpropyl)oxan-4-ol) | 150 | 150 |
| Ethylene brassylate (1,4-dioxacycloheptadecane-5,17- | 170 | 170 |
| dione) | | |
| Galaxolide 50% IPM (1,3,4,6,7,8-hexahydro-4,6,6,7,8, | 200 | 200 |
| 8-hexamethylcyclopenta(g)-2-benzopyran 50% in | | |
| isopropyl myristate) | | |
| compound P | 20 | 50 |
| Total ( part by weight) | 1020 | 1050 |

Composition according to table II and table III namely 1A, 1B, 2A,2B could be included in various compositions selected from, Deo pump spray, Clean hair-conditioner, Face wash gel, Foam bath concentrate, Hair gel, Self-foaming bodywash, Sprayable sun care emulsion, Sprayable sun protection emulsion, Emollient facial gel, 2-phases oil foam bath, Shampoos, Shower bath, Hydro-alcoholic AP/Deo pump spray, Aerosol,

Aqueous/alcoholic AP/Deo roll-on, Styling Gel Type “Out of Bed”, Shaving Foam, Sensitive skin Baby shampoo, Body wash for Sensitive Skin, Gloss Enhancing Shampoo for Sensitive Scalp, Deo Stick, Baby Wipe, After shave balm, Face Gel, Face Day Care Cream, Face Cleanser, Body lotion, Sun Care SPF50+, Sprayable Lotion, Hand dish cleaner—regular, Hand dish cleaner—concentrate, Sanitary cleaner—concentrate, All-purpose cleaner, Anti-bacterial fabric softener, Detergent composition, Powder detergent composition and Liquid detergent composition.

A person skilled in art may be well versed with the various formulations for the above-mentioned products.

Compositions 1A, 1B, 2A and 2B can for example be formulated in specific formulations as disclosed in IP.com Number: IPCOM000258614D entitled New Aroma Chemicals pages 6 to 46, Table 1 to Table D13, wherein the “Fragrance Composition 1A” is replaced by identical amounts of compositions 1A, 1B, 2A or 2B.

1.-26. (canceled)

27. An aroma chemical comprising at least one compound of formula (I) 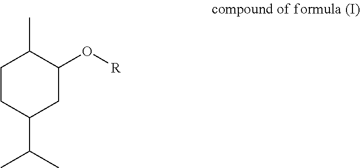
or of a stereoisomer thereof or of a mixture of different stereoisomers thereof, wherein
---- represents a single bond or a double bond, and
R is linear or branched C₁-C₆-alkyl, formyl, or linear or branched C₁-C₄-alkylcarbonyl.

28. The aroma chemical according to claim 27, wherein the compound of formula (I) is compound of formula (Ia), 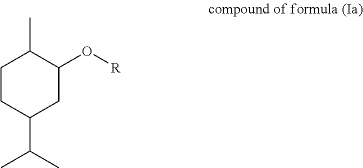
or of a stereoisomer thereof or of a mixture of different stereoisomers thereof, wherein
R is linear or branched C₁-C₆-alkyl, formyl, or linear or branched C₁-C₄-alkylcarbonyl.

29. The aroma chemical according to claim 27, wherein the compound of formula (I) is compound of formula (Ib) 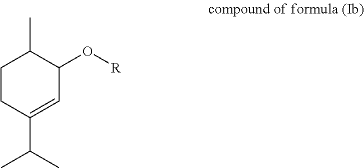
or of a stereoisomer thereof or of a mixture of different stereoisomers thereof, wherein
R is linear or branched C₁-C₆-alkyl, formyl, or linear or branched C₁-C₄-alkylcarbonyl.

30. The aroma chemical according to claim 27, wherein R is methyl, ethyl, propyl, isopropyl, n-butyl, isobutyl, tert-butyl, pentyl, hexyl, methylcarbonyl, ethylcarbonyl, n-propylcarbonyl, isopropylcarbonyl, n-butylcarbonyl, isobutylcarbonyl or tert-butylcarbonyl.

31. The aroma chemical according to claim 27, wherein the compound of formula (I) is a compound of formula (Ia), wherein R is linear or branched C₁-C₃-alkylcarbonyl.

32. The aroma chemical according to claim 27, where the compound of formula (I) is used in admixture with a compound of formula (II) 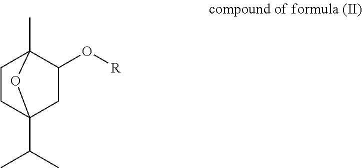
or a stereoisomer thereof or a mixture of different stereoisomers thereof, wherein
R is as defined in claim 27.

33. A method of imparting an aroma impression to a composition, comprising at least the step of adding at least one compound of formula (I) according to claim 27 to a composition to be aromatized, or mixing at least one compound of formula (I) according to claim 27 simultaneously or consecutively with the other components of said composition, or mixing at least one compound of formula (I) according to claim 27 with a pre-formed mixture of a part of the other components of said composition.

34. The method according to claim 33, wherein the aroma impression is selected from a fresh note, minty note, moldy note, carrot like note, natural note, rooty note, violet note, powdery note, floral note, woody note, warm note, ionone note, fruity note, camphoraceous note, citrus note, herbal note, ethereal note, gourmand note, anisic note, ambrinol like note, animalic note, civet note, soft note, incense note, musty note, fatty note, tobacco note, green note, spicy note, agrumic note, juniper note, pepper note, spearmint note, cedarwood note, dandelion note, earthy note, food-like note, or a combination of two or more of these notes.

35. The method according to claim 33, wherein the composition is selected from perfume compositions, body care compositions, hygiene articles, cleaning compositions, textile detergent compositions, compositions for scent dispensers, foods, food supplements, pharmaceutical compositions, or crop protection compositions.

36. The method according to claim 33, wherein the compound of formula (I) (if no compound (II) is present) or the mixture of compounds (I) and (II) is present in a total amount in the range of ≥0.01 wt.-% to ≤70.0 wt.-%, based on the total weight of the composition.

37. A compound of formula (Ia) 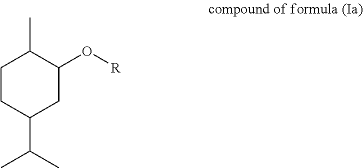
or a stereoisomer thereof or a mixture of different stereoisomers thereof,
wherein R is isopropylcarbonyl.

38. A compound of formula (Ib) 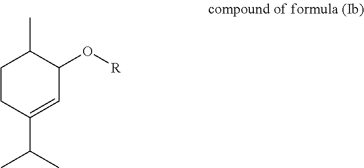
or a stereoisomer thereof or a mixture of different stereoisomers thereof,
wherein
R is linear or branched C₁-C₆-alkyl.

39. The compound of formula (Ib) according to claim 38, wherein R is methyl or ethyl.

40. A composition comprising at least one compound of formula (I) according to claim 27 and
(i) at least one aroma chemical (X) different from the compounds of formula (I) and (II), or
(ii) at least one non-aroma chemical carrier, or
(iii) both of (i) and (ii).

41. The composition according to claim 40, comprising at least one compound of formula (I), at least one aroma chemical (X) different from the compounds of formula (I) and (II), and optionally at least one non-aroma chemical carrier, where the at least one aroma chemical (X) different from the compounds of formula (I) and (II) is selected from the group consisting of: geranyl acetate, alpha-hexylcinnamaldehyde, 2 phenoxyethyl isobutyrate, dihydromyrcenol, methyl dihydrojasmonate, 4,6,6,7,8,8 hexamethyl-1,3,4,6,7,8-hexa-hydro-cyclopenta [g]-benzopyran, linalool, tetrahydrolinalool, ethyllinalool, benzyl salicylate, 2 methyl-3-(4-tert-butylphenyl) propanal, cinnamyl alcohol, 4,7-methano-3a,4,5,6,7,7a-hexahydro-5 indenyl acetate, 4,7-methano-3a,4,5,6,7,7a-hexahydro-6-indenyl acetate, cit-ronellol, citronellyl acetate, citronellol, geraniol, tetrahydrogeraniol, vanillin, linalyl acetate, styrolyl acetate, octahydro-2,3,8,8-tetramethyl-2-acetonaphthone, 2-acetyl-1,2,3,4,6,7,8-octahydro-2,3,8,8-tetramethyl-naphthalene, hexyl salicylate, 4-tert-butylcyclohexyl acetate, 2-tert-butylcyclo-hexyl acetate, alpha-ionone, n-alpha-methylionone, alpha-iso-methylionone, coumarin, terpinyl acetate, 2-phenylethyl alcohol, 4-(4-hydroxy-4-methylpentyl)-3-cyclohexenecarboxaldehyde, alpha-amylcinnamaldehyde, ethylene brassylate, (E)-3-methylcyclopentadec-5-enone, (Z)-3-methylcyclo-penta-dec-5-enone, 15-pentadec-11-enolide, 15-pentadec-12-enolide, 15-cyclo pentadecanolide, 1-(5,6,7,8-tetrahydro-3,5,5,6,8,8-hexamethyl-2-naphthalenyl) ethanone, 2-isobutyl-4-methyltetrahydro-2H pyran-4-ol, 2-ethyl-4-(2,2,3-trimethyl-3-cyclopenten-1-yl)-2-buten-1-ol, cis-3-hexenyl acetate, trans-3-hexenyl acetate, trans-2/cis-6-nonadienol, 2,4-dimethyl-3-cyclohexenecarbox-alde-hyde, 2,4,4,7-tetramethyloct-6-en-3-one, 2,6-dimethyl-5-hepten-1-al, borneol, 3-(3-isopropylphenyl) butanal, citral, citronellal, 2-methyl-3-(3,4-methylenedioxyphenyl)-propanal, 3-(4-ethylphenyl)-2,2-dimethylpropanal, 7-methyl-2H-1,5-benzodioxepin-3 (4H)-one, 3,3,5-trimethylcyclohexyl acetate, 2,5,5 trimethyl-1,2,3,4,4a,5,6,7-octahydronaphthalen-2-ol, 3-(4-tert-butylphenyl)-propanal, ethyl 2-methylpentanoate, ethoxymethoxycyclododecane, 2,4-dimethyl-4,4a,5,9b-tetrahydroindeno[1,2-d][1,3]dioxine, (2-tert-butyl-cyclo-hexyl)acetate, 3-[5,5,6-trimethylbicyclo[2.2.1]hept-2-yl]cyclohexan-1-ol, menthone, isomenthone, carvone, camphor, beta-ionone, beta-n-methylionone, beta-isomethylionone, alpha-irone, alpha-damascone, beta-damascone, beta-damascenone, delta-damascone, acetylated cedar wood oil, and mixtures thereof.

42. The composition according to claim 40, wherein the non-aroma chemical carriers are selected from the group consisting of surfactants, emollients and solvents, where the solvents are selected from the group consisting of ethanol, isopropanol, propylene glycol, dipropylene glycol, 1,2-butylene glycol, diethylene glycol monoethyl ether, glycerol, diethyl phthalate, isopropyl myristate, triethyl citrate, and benzyl benzoate.

43. The composition according to claim 40, which is selected from perfume compositions, body care compositions, hygiene articles, cleaning compositions, textile detergent compositions, compositions for scent dispensers, foods, food supplements, pharmaceutical compositions, or crop protection compositions.

44. A mixture comprising at least one compound of formula (I) 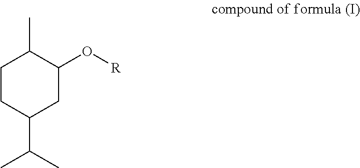
or a stereoisomer thereof or a mixture of different stereoisomers thereof,
where
---- represents a single bond or a double bond,
and
at least one compound of formula (II) 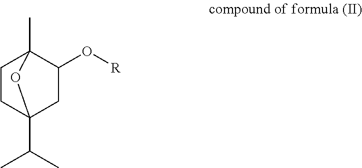
or a stereoisomer thereof or a mixture of different stereoisomers thereof,
where
R in compounds (I) and (II) is independently as defined in claim 27.

45. The mixture according to claim 44, wherein R is methyl, ethyl, methylcarbonyl, ethylcarbonyl or isopropylcarbonyl.

46. The mixture according to claim 44, wherein the weight ratio of the total amount of the compounds of formula (I) and the total amount of the compounds of formula (II) is in the range from 99.99:0.01 to 0.01:99.99.
